SEQUENCE LISTING (21 sequences)

SEQ ID NO: 1 - DNA, 1461 bp, Cucumber fruit mottle mosaic virus
tcgaaggcgggtttttggacggatatgcaaaatttttatgacgcttgtctgcccgggaatagttttgtgttgaatgattacgattctgtgactatgcggctggttgataatgagattaatctgcaaccttgtaggttaactctatctaaagccgatcccgttacagagtctctgaagatggagaaaaaggagtttttgatcccgcttggtaaaactgctacggagcgtccgcggatccctgggcttttagaaaatttgatagctatagttaagaggaattttaatacaccggatttagccgggagtttagatatttctagtattagtaagggtgtagtagataacttcttttccacttttttgcgtgacgagcaattggcggatcacctttgtaaagttaggtctcttagtctagagtctttttccgcatggtttgataatcaatcaacttgtgctctgggtcagttgtctaatttcgattttgtggatctgcctcccgttgatgtttataatcatatgattaagaggcaacccaaatcgaagttagacacctcgattcagtctgagtatcccgcgttgcaaacgattgtttatcatagtaaattagtgaatgcggtttttggtcccgttttccgttatcttacttccgagtttttatctatggtagataatagtaaatttttcttttatactagaaaactccggatgatttgcaagtttcttttcccacactttcccaataagcaggagtatgagattctagagctagatgtttccaaatatgataaatcacagaatgattttcatcaggctgtggagatgcttatttgggaacgtttaggtctagatgatattcttgctaggatttgggaaatggggcataagaagacgcatatcagtgatttccaagctgggattaaaactcttatttattatcagcggaaatctggagatgttactacttttataggtaatacttttattatagctgcttgtgtcgcttctatggttccgctgagtcggagtttcaaagctgccttttgtggtgatgattcactgatctatatgccaccgaatctggaatataatgacatacagtcgaccgcgaatctcgtgtggaatttcgaggctaaactgtataagaagaaatatggttatttctgtggcaaatatgtgatccatcatgcgaatgggtgtattgtttatccggatccgttgaagctaatttctaaattaggcaataagagtctggaaagttacgatcatttggaggaatttaggatttctctgatggacgtagctaaacctttgttcaatgctgcttattttcatcttttagatgatgctatccacgagtattttcctagtgttgggggtagcacgtttgctattagttctttgtgcaagtatcttagtaataagcagttgtttgggtctctattcattaagcctagtgtctag

SEQ ID NO: 2 - DNA, 1050 bp, Zucchini yellow mosaic virus
tcaggcactcagccaactgtggcagacactggagccacaaagaaagacaaagaagatgacaaagggaaaaacaaggatgttacaggctccggctcaagtgagaaaacagtggcagctgtcacgaaggacaaggatgtaaatgctggttctcatgggaaaattgtgccgcgtctttcgaagataacaaagaagatgtcactgccacgcgtgaaaggaaatgtgatactcgacattgatcacttgctggagtataagccggatcaaattgagttatacaacacacgagcgtctcatcagcaattcgcctcttggttcaaccaagttaaaacagaatatgatctgaatgagcaacagatgggagttgtaatgaatggtttcatggtttggtgcatcgaaaatggcacgtcacccgacattaacggagtatgggttatgatggacggtaatcagcaggttgaatatcctttgaaaccaatagttgaaaatgcaaagccaacgctgcgacaaataatgcatcacttttcagatgcagcggaggcatatatagagatgagaaatgcagaggcaccatacatgccgaggtatggtttgcttcgaaacttacgggataggagtttggcacgatatgctttcgacttctacgaagtcaattccaaaactccggaaagagcccgcgaagctgttgcgcagatgaaagcagcagcccttagcaatgtttcttcaaggttgtttggccttgatggaaatgttgccaccactagcgaagacactgaacggcacactgcacgtgatgttaataggaacatgcacaccttgctaggtgtgaatacaatgcagtaaagggtaggtcgcctacctaggttatcgtttcgctccgacgtaattctaatatttaccgctttatgtgatgtctttacatttctagagtgggcctcccacctttaaagcgtaaagtttatgttagttgtccaggagtgccgtagtcctgtcggaagctttagtgtgagcctctcacgaataagctcgagattagactccgtttgcaagcct

SEQ ID NO: 3 - DNA, 190 bp, Ricinus communis
gtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacag

SEQ ID NO: 4 - DNA, 6562 bp, Cucumber fruit mottle mosaic virus
gataaaagttttttacattgaacaaaaacaatatacattacttttataacaatacacaatacatggcaaacattacacaacatatcaatgataccagggaggctgcggccgccgggcgtaatccgctcgtggcgcagctggcttcgaagagggtttatgatgaggctgtcaagtctcttgattctcaagataaacgccctaaggtgaattttgctcgggtattgaccacagagcagacgaggaaggtcacggagtcgtatccggagttttcgatcagttatactgcatcggccttatctgtgcatagtttggcggggggactgcgatatttagaaggtgagtacctgatgatgcaggttccctatgggtcgcccgtgtatgatatcggagggaattactcgcaacatatgctgaaggggagagcatacgtacattgttgcaatccgtgcctggacctgaaggacattgcacgcaatgagatgtacaaggatgccattgaccgttatgtgcataagaaacgcgaagcgccacgttctaatgcttggagggctagggcagagtccgtccaagaaattaaagacggccgtctaccttcatggcagatcgatgcgtttcagcgatataaggattgtccaagagcggtcacctgtaatgatgtgttccaagagtgtcagtatgaacatacgaggagaggggatcgttatgcagttgctctgcattcgatttatgatattcctttcgaacagataggacctgcgctcttgcggaagaatattaaggttctcttcgccgcattccatttctcagaggagttgctgttggggcaaagttttggtgccttgcctaatataggtgcgttctttaccgtcaatggtgattccgtcgagtttcagttcgaagaagaatctactttgcattattcacatagtttccagaatattaggaagatagtaactaggacgtattttcctgcttcagatagggtagtttatgtaaaggagtttatggttaagcgtgtagatactttctttttccgtatggttagggttgatacccatatgttacataagtcagtaggtacgtatcctgtttgtgcgactaactatttctctctcaagtcatcaccaatattccaggataaagccacgttctctgtgtggtttcccaaagctaaatctaaggtggtgatacctatctttaagatgcaagggtttttcactgggtctattgtggcagagaagatgatgatcgatgctagctttattcatactgttatcaatcatatctgtacttatgataataaggcgttaacgtggaggaatgttcagtccttcgtcgagtcaattcggtcccgggttgtcgtgaatggggtttcggtgcggagtgaatgggatgtgccggtagagcttttaactgatatttcgttcaccgtttttttactagtcaaagtcaagaagacgcagatcgagattatgagtgataaaattgtgacacaacctcaggggttgattgagcggattgtacagagagtctctgaagctttcgaaggatgtacagaagcggtgcaaaaggcccttcttacttccgggtggttcagaactccagcggatgatctcgttcttgatattcctgagttgttcatggattttcatgattatctcagcggtgtcttcgaaagccggatgctcgtattgaggcgacggaccgtcgaaaaatgttttaagcgctttccgacaagctttattcgactgtatcggaagctttgtgagcgatattctgggattgaatttgacttggagcaagtttctgatttttgccaccaccatgacgtgaatcctgctttggtgggacccgtgatagaggcgattttttcgcagactgccgggattacagtcactgggctgtctacaaaatctgttgagtgggcagccgcagaggctttagcaccgacgtctgttgatatggattgtgacagtgatgatgaggagctggagcagaaattcccaaatctgtccaatgaggagttgagatatttgcatgaggtgagatcgaaggaagccgctttcttggagctacaagatacatttaaaaccaagaaggtgactgagttagtgtctgtgggagtaggagctttgccaacgctaccgcgtcagtggatagcgacagggaaggttcatcttcctcaggttggtctgtcggttgggaagaataaacattcggtcgagatatgtgacgaagatggggtcagtgtgaagaatctgcatctgacggagacgtgtaatctaagattgaagaagactatcactccggtgatctatactgggcccataagagtgcgtcagatggctaattatctcgattatctttctgctaatctggccgctacgataggaattctcgaaagaattgttcgatcgaattggtctgggaatgaggttgtgcaaacttatggtctttttgattgtcaggctaataagtggatcttactgccctctgagaaaacacatagttggggtgtctgtctgactatggatgataagcttcgtgttgtcctgctgcagtatgattccgccggttggccgattgtagataagtctttttggaaagctttttgtgtgtgtgcggatactaaagttttttctgttattaggagtcttgaggttttgtctgctttacctttagttgaaccggatgctaagtatgtgctgattgatggtgtgcctggttgtgggaagacgcaagagattatatcgagtgcggacttcaaaacggatctaatccttacacctggtaaggaagccgcggccatgatcaggcgtagagccaacatgaaatataggagtcccgtcgccacaaatgataatgtgaggacttttgattcatttgtaatgaataaaaagccctttacctttaagacactatgggtggatgagggtctcatggtgcataccggtctgttaaatttctgtgtgaatattgctaaggtaaaggaagttcgtattttcggtgatactaagcaaatccccttcattaatagagtgatgaatttcgattacccactagagctgaggaaaattattgttgatacggtggaaaagcggtacacgagtaaacggtgtccaagggatgtgactcattatttgaatgaggtatattccagtcccgtgtgtactactagtcctgtcgtacattcagttactacaaaaaagattgctggagtgggtcttttgcgaccggaattgacggcattgcctggtaagattataactttcactcagaatgacaagcaaacgcttttaaaagcgggttatgctgatgtgaatactgtgcatgaggtgcagggggagacatatgaggaaacttccgtggtgagggctactgctacaccaattggtttgatttcgcgtaagtctccgcatgtgcttgttgctctgtcgaggcataccaaggcgatgacgtattatactgtgactgtggatcccgtgagctgtataattgctgatttggagaaggtcgatcaaagtattctgtctatgtatgcctctgtggcggggaccaaatagcaattacagcaactatccgtctatgtgcatttgcccgtgtcgaaggcgggtttttggacggatatgcaaaatttttatgacgcttgtctgcccgggaatagttttgtgttgaatgattacgattctgtgactatgcggctggttgataatgagattaatctgcaaccttgtaggttaactctatctaaagccgatcccgttacagagtctctgaagatggagaaaaaggagtttttgatcccgcttggtaaaactgctacggagcgtccgcggatccctgggcttttagaaaatttgatagctatagttaagaggaattttaatacaccggatttagccgggagtttagatatttctagtattagtaagggtgtagtagataacttcttttccacttttttgcgtgacgagcaattggcggatcacctttgtaaagttaggtctcttagtctagagtctttttccgcatggtttgataatcaatcaacttgtgctctgggtcagttgtctaatttcgattttgtggatctgcctcccgttgatgtttataatcatatgattaagaggcaacccaaatcgaagttagacacctcgattcagtctgagtatcccgcgttgcaaacgattgtttatcatagtaaattagtgaatgcggtttttggtcccgttttccgttatcttacttccgagtttttatctatggtagataatagtaaatttttcttttatactagaaaactccggatgatttgcaagtttcttttcccacactttcccaataagcaggagtatgagattctagagctagatgtttccaaatatgataaatcacagaatgattttcatcaggctgtggagatgcttatttgggaacgtttaggtctagatgatattcttgctaggatttgggaaatggggcataagaagacgcatatcagtgatttccaagctgggattaaaactcttatttattatcagcggaaatctggagatgttactacttttataggtaatacttttattatagctgcttgtgtcgcttctatggttccgctgagtcggagtttcaaagctgccttttgtggtgatgattcactgatctatatgccaccgaatctggaatataatgacatacagtcgaccgcgaatctcgtgtggaatttcgaggctaaactgtataagaagaaatatggttatttctgtggcaaatatgtgatccatcatgcgaatgggtgtattgtttatccggatccgttgaagctaatttctaaattaggcaataagagtctggaaagttacgatcatttggaggaatttaggatttctctgatggacgtagctaaacctttgttcaatgctgcttattttcatcttttagatgatgctatccacgagtattttcctagtgttgggggtagcacgtttgctattagttctttgtgcaagtatcttagtaataagcagttgtttgggtctctattcattaagcctagtgtctagatgtccatcagtaaggtcggtgtcaggaacgctttaaagccagaggaatttgttaagattacttgggttgataagctacttcctgatgcttttactattcttaagtatttatctattacagattatagtgttgtacagtctaaagactatgaacatctcatacctgtggatctactacgtggcgtggatttttcaaagtctaaatatgttactttggttggtgttgtgatctccggggtctggacaattcctgagaattgtgccggtggtgctaccgtggcgctggtcgatactcggatgtccttagtgtcggagggtactatttgtaagttttctgtatctgcagccagtcgggattttacggtaaaattgattccgaattattatgtgactgctgctgatgcctcctccaaaccctggtctttgtttgttaggatttctggtgttaggatcaaagatggtttttctccgttaactctggagattgcctctttggtggctaccaccaattctattttaaagaagggtctaagagttagtgtgatcgagtccgtggtagggtctgatgcctccgtgtcgttggataccttgtctgaaaaggttcaacccttttttgattcggttccgattacggcttcagtggtatctcgtgataggtcttatgtgtctaaaggtcgccctccttctcggagtggacctgtgtcgcgtaaatctaaaagtaagagtgaggcagaatctttttcggatagcggcgcttctgagccactaagttcataatcaagatgtcttactctacttctggtttgcgttctttgcctgcatatactaagtctttttgtccttattatgctttgtatgatctgttggtgtcagcccaaggtggagccctgcaaacgcaaaatggtaaagacattttgcgtgactccataaatgggttgttaacgacagttgcgtctcctaggagtcggttccctgcggaagggttctttgtctggtcccgtgagtcgcgcattgctgctatattagattctctgctttcggcgttggattcaagaaatagggctattgaagttgaaaacccttctaatccttcgaccagtgaagctttgaatgctactaagcgcaatgacgacgcgtctactgctgcgcacaacgacattcctcagctgatttcagctttgaatgacggtgccggtgtgtttgatagagcgtcttttgagagtcagttcggtctagtatggaccgctgcgtcgtcgtctacctcgaagtgaggcgtggtcgctgcgttaagcgatagagtttttccctcctctttaatcgaagggtttcccgttatgggcgtggtcatcacgaaagatgatagagtttttccctcctcttaaatcgaagggattgtttgcgcggtttctaccgagcctctgctgtgtgacagtaagctggcgtaagcaattatgggtagaggtgttcgaatcaccccctttgccccgggtaggggccca

SEQ ID NO: 5 - DNA, 25 bp, Artificial sequence
  Single strand DNA oligonucleotide
cgctagctatcactgaaaagacagc

SEQ ID NO: 6 - DNA, 26 bp, Artificial sequence
  Single strand DNA oligonucleotide
ggccatggttatgtctgaagtaaacg

SEQ ID NO: 7 - DNA, 32 bp, Artificial sequence
  Single strand DNA oligonucleotide
cggccatggcatcgaaggcgggtttttggacg

SEQ ID NO: 8 - DNA, 32 bp, Artificial sequence
  Single strand DNA oligonucleotide
gaggtgacctagacactaggcttaatgaatag

SEQ ID NO: 9 - DNA, 9591 bp, Zucchini yellow mosaic virus
aaattaaaacaaatcacaaagactacagaaatcaacgaacaagcagacgatttctaaaccgtgtttacaaacaagcaatctataattcgcacagcatcaagaatttctgcaatcactttgtttattttagacacaacaatggcctcagttatgattggttcaatctccgtacctatcgcacaacctgcgcagtatgcaaacacccaagcgagcaaccgggttaatatagtggcacctggccacatggcaacatgcccaccaccattgaagacgcacacatactacaggcatgagtccaagaagttgatgcaatcaaacaaaagcattgacattctgaacaacttcttcagcactgacgagatgaagtttaggctcactcgaaacgagatgagcaaggtgaaaaagggtccgagtgggaggatagtcctccgcaagccgaataagcagcgggttttcgcccgtattgagcaggatgaggcagcacgcaaggaagaggctgttttcctcgaaggaaattatgacgattcgatcacaaatatagcacgtgttcttccacctgaagtgactcacaacgttgatgtgagcttgcgatcaccgttttacaagcgcacatacaagaaggaaagaaagaaagtggtgcaaaagcaaactgtgctagcaccacttaatagtttgtgcacacgtgttctgaaaattgcgcgcaataaaaatatccccgttgagatgattggcaacaagaaggcgagacatacactcaccttcaagaggtttaggggatattttgttggaaaagtgtcagttgcgcatgaagaaggacgaatgcggcatactgagatgtcgtatgagcaatttaaatggattctaaaagccatttgtcaggtcacccatacagagcgaattcgtgaggaagatattaaaccaggctgtagtgggtgggtgttaggcactaatcatacattgactaaaagatattcaagattgccacatttggtgattcgaggtagagacgacgacgggattgtgaacgcgctggaaccggtgttgttttatagcgaagttgaccactattcgtcgcaaccggaagttcagttcttccaaggatggcgacgaatgtttgacaagcttaggcccagcccagatcatgtgtgcaaagttgactacaacaacgaggaatgtggtgagttagcagcaaccttttgtcaggctctattcccagtagtgaaactatcgtgccaaacatgcagagaaaaacttagtagagttagctttgaggaatttaaagattctttgaatgcaaactttactattcataaagatgaatgggataatttcaaggagggctctcagtacgacaatattttcaaattaattaaagtggcaacacaggcaactcagaatctcaagctctcatctgaagtcatgaagttagttcagaaccatacaagcactcacatgaagcaaatacaagatatcaacaaggcgctcatgaaaggttcattggttacgcaagacgaattggacttggctttgaaacagcttctagaaatgactcagtggtttaggaaccacatgcacctgactggtgaagaagcgttgaagatgttcagaaacaagcgctctagcaaggctatgataaatcctagccttttatgtgataaccaattggacaagaatggaaattttgtctggggggaaagagggtatcattccaagcgattattcaagaacttctttgaggaagtaataccaagtgaaggatatacgaaatacgtagtgcgaaactttccaaatggtactcgtaagttggccataggctctttgattgtaccactcaacttggatagggcacgcactgcacttcttggagagagtattgagaagaagccgctcacatcagcatgtgtctccaaacagaatggaaattatatacactcatgctgctgcgtaacgatggatgatggaaccccgatgtactcagaacttaagagcccaacgaagaggcacctagttataggagcttctggtgatccaaagtacattgatttaccagcatctgaggcagaacgcatgtatatagcaaaggaaggttattgctatcttaacattttcctcgcaatgcttgtgaatgttaacgagaacgaggcaaaggatttcactaaaatgattcgtgatgttttgatccctatgcttgggcaatggccttcgttgatggatgttgcaactgcagcatacattctaggtgtattccatcctgaaacacgatgtgctgaattacctaggattcttgttgatcaccgtacgcaaaccatgcatgtcattgattcatatggatcattaactgttggttatcacgtgctcaaggccggaactgtcaatcatttaattcaattcgcctcaaatgatctacagagcgagatgaagcattacagagttggcggaacaccaacacagcgcatcaaacttgaggagcagttgattaaaggaatcttcaaaccaaaacttatgatgcagctcctgcacgatgatccatacatattattgcttggcatgatctcacctaccattcttgtacacatgtataggatgcgtcattttgagcgaggtattgaaatatggattaagagagatcatgaaattggaaagattttcgtcatattggaacagctcacacggaaggttgctctggctgaagttcttgtggatcagctcgatttgataagcgaagcttcaccacatttacttgaaatcatgaagggttgtcaagataatcaaagagcgtatgtacctgcgctggatttactaacgatacaagtagagcgtgagttttcaaacaaggaacttaaaaccaatggctatccagatttgcagcaaacgttgtttgatatgagagaaaaaatgtatgcaaagcaattgcacaatgcatggcaagagctaagcttgctggaaaaatcttgtgtaaccgtgcgattgaagcaattctcgatttttacggaaagaaatttaatccagcgagcaaaagaaggaaagcgcacatcttcgctacaatttgttcacgagtgctttatcacgacccgagtacatgcgaagagcattcgcgatgcaggcgtgcgcaagctaaatgaggctctcgttggtacttgtaagttctttttctcttgtggtttcaagatttttgcgcgatgctacagcgacattatataccttgtgaacgtgtgtttggtattctccttggtattacaaatgtctaatactgtacgcaacatgatagcggcgacaagggaagaaaaagagagagcgatggcaaataaggctgatgaaaatgaaaggacgttaatgcacatgtaccacattttcagtaagaagcaggatgaagcgcccatatacaacgactttcttgagcatgttcgcaatgtgagaccagatcttgaggaaaccctcttatacatggctggtgcagaggttgtagcaacacaggccaagtcagcggttcagattcagttcgagaagattatagctgtattggcgctgctcactatgtgctttgacgccgaaagaagcgacgccattttcaagattttgacaaagctcaaaacagtttttggcacggttggagaaacggtccgacttcaaggacttgaagatattgagagcttggaggacgataagagactcacaattgactttgatattaacacgaatgaggctcagtcgtcgacaacgtttgatgttcattttgatgattggtggaatcggcagctgcagcaaaatcgcacagttccacattacaggaccacaggtaaattcctcgaatttaccagaaacactgcagcttttgtggccaatgaaatagcatcatcaagtgaaggagagtttttagtcagaggagcagtgggttctggaaaatcaacgagcttgcccgcacatcttgccaagaagggtaaggtactgttacttgaacctacacgccccttggcggagaatgtcagtagacagttggcaggcgatccttttttccaaaacgtcacactcagaatgagagggctaaattgctttggttcaagtaacattacagtgatgacgagtggatttgcttttcactattatgttaacaatccacatcaattaatggaatttgactttgttataatagacgagtgccatgtcacggacagtgcgactatagctttcaattgtgcgcttaaggagtataactttgctggcaaattgattaaagtgtctgcaacgccgccagggagagagtgtgatttcgatacgcaattcgcggttaaagtcaaaacggaggaccacctttcattccatgcattcgttggcgcacagaagaccggttcaaatgctgacatggttcagcatggcaataacatacttgtgtatgttgcaagttacaacgaagtggacatgctttccaagttactcactgagcgacaattttcagtgacgaaggtagatgggcgaacaatgcaacttgggaaaactaccattgaaacgcatggaactagccaaaagcctcatttcatagtagctagaaacatcatcgaaaatggagtgacgttggatgttgagtgtgttgttgattttggactgaaagtggtcgcagaattagacagcgaaaatcggtgtgtgcgctacaacaagaaatcagttagttatggggaaaggattcagcggctagggagagtggggagatctaagcctggaacggcattgcgtatagggcacacagaaaaaggcatcgagagcattcctgaattcattgccacagaagcagcagccctatcgtttgcctatgggcttccagtcactacgcatggggtttccacaaatatactcggaaagtgcacagtcaagcagatgagatgtgctttgaatttcgagctaactcctttcttcaccactcatctaatccgtcatgatggcagtatgcacccattgatacacgaagaattaaaacaattcaaactcagggattcagaaatggtgctcaacaaggttgcattacctcaccaatttgtgagtcaatggatggatcaaagtgagtatgaacgcattggagtgcacgttcaatgtcatgagagcacacgcatacctttctacacaaatggagtgcctgacaaggtctatgagaaaatttggaagtgcatacaagaaaacaagaatgatgcggtttttggtaagctctcaagcgcttgttcgactaaggtcagttatacactcagcactgacccagcagcattacccagaaccattgcaatcatcgaccacctgcttgccgaggaaatgatgaagcggaatcacttcgacacgattagctcagctgtgacgggttattcattttccctcgctggaattgctgattcttttaggaagagatacatgcgtgattacacagcgcacaacattgcaattcttcaacaagcacgtgcccagctgctcgagttcaatagcaaaaatgtgaacatcaacaacctgtccgatctggaaggaattggagttattaagtcggtggtgttgcaaagtaaacaagaggtcagcaacttcttaggacttcgcggtaaatgggatggacggaaatttgcgaacgatgtgatattggcgattatgacactcttaggaggtggatggttcatgtgggaatacttcacgaaaaagatcaatgaacctgtgcgcgttgaaagcaagaaacggcgatctcaaaagttgaaattcagggatgcatacgataggaaggtcggacgtgagatctttggcgatgatgacacaattgggcgcacttttggcgaagcttacacgaagagaggaaaggtcaaaggaaacaacagcacaaaaggaatgggacggaaaactcgcaattttgtgcatttatatggtgtggagcctgagaattacagcttcatcagatttgtggaccctctcactggccatacattggacgaaagcacccatacagacatatcgttagtgcaggaggagtttggaaatattagagagaaatttctggagaatgatttgatctcaaggcagtctattatcaacaaacccggtattcaggcatattttatgggcaagggcactgaagaagcactcaaagttgatttgactcctcatgtaccattgcttctgtgcaaaaacaccaatgccattgcgggatacccagagagagaaaatgagctgagacaaactggcacaccagtcaaggtttcttttaaagacgtgccagagaaaaacgaacatgtcgagttggagagcaaatccatctacaaaggagtgcgcgattataatggcatctcaacaatcgtctgtcaattaacgaatgattctgatggcctcaaggagactatgtatggtattggctatgggccgataatcatcactaatggacacctcttcaggaaaaacaatggcacacttctagtcaggtcttggcatggtgaattcactgtaaaaaataccacaacgctcaaagtgcatttcatagaaggaaaggatgttgttttagtgcgtatgccaaaggactttccaccgtttaaaagcaacgcttcttttagggcaccaaagcgcgaggaacgagcatgtttggttggaacgaattttcaagagaagagtctccgctccactgtttcagaatcttccatgacaatacctgaaggaactggctcatattggattcactggatttcgaccaacgaaggggattgcggattacccatggtttcaacaacggatggcaagataattggagttcatggtttggcttccacggtctcatccaagaattattttgtcccatttactgatgattttatagccacgcatttgagcaagcttgacgatctcacatggactcaacattggctatggcaacctagtaaaatcgcgtggggcacgcttaacttagttgatgaacaaccagggcctgaatttcgtatttcaaatctagtaaaggatttgttcacttctggtgttgaaacacagagcaagcgagaaagatgggtctacgaaagctgtgaagggaaccttcgagctgttggaactgcacaatcagcgctagtcaccaaacatgttgtaaaaggcaagtgtcctttcttcgaagaatacttgcaaactcacgcagaagcgagcgcttacttcagacccttgatgggagaataccagcctagcaagttgaacaaagaggcctttaaaaaggatttcttcaaatacaataaacccgtcactgttaatcaattggatcacgataaattcttggaagcagttgatggggttatacgtatgatgtgcgactttgaattcaatgagtgccgattcattacagaccccgaggaaatttataactctttgaacatgaaagcagcaattggagcccagtatagaggaaagaagaaagagtattttgaagggctagatgattttgatcgagagcgacttttatttcagagttgtgaaaggttgtttaatggctacaaaggtctgtggaatggatctttaaaggccgagctcaggccgcttgagaaagtcagggccaacaaaacacgaacttttacagcagcgcccattgatacattgctcggagctaaagtttgcgtggatgatttcaataatgaattttatagcaaaaacctcaagtgtccatggacggttggcatgacgaaattttatggtggttgggatagattgatgagatcattacctgatggttggttatattgtcatgctgatggatcacagtttgacagttcattgaccccagccttactgaatgcagtgcttataatccgatcattttatatggaggattggtgggtcggtcaagagatgcttgaaaatctttatgctgagattgtgtacactccaattcttgctccggatggaacaattttcaagaaatttagaggtaacaacagtgggcaaccctcaacagtggtggataacacactaatggttgtgatctctatttactatgcgtgcatgaaatttggttggaattgcgaggaaattgagaatagacttatcttctttgcaaatggagatgatctgatacttgcagtcaaagatgaggatagcggcttacttgataacatgtcagcttctttttccgaactcggactgaattatgatttttcagaacgcacgcacaaaagagaagatctttggttcatgtcccaccaagcaatgttagttgatggaatgtacattccaaaactcgagaaagaaagaattgtttcaattctagagtgggatagaagcaaagaaatcatgcaccgaacagaggctatttgcgctgcgatgattgaggcatgggggcacaccgagcttttgcaagaaatcagaaagttttacctatggttcgttgagaaagaggaagtgcgagaattagctgccctcggaaaagctccatacatagctgagacagcacttcgcaagttatatactgacaaaggagcggaaacaagtgaattggcacgctacctacaagccctccatcaagatatcttctttgaacaaggagacaccgtaatgctccaatcaggcactcagccaactgcggcagacgctggggccacaaagaaagacaaagaggatgacaaagggaaaaacaaggatgttacaggctccggctcaggtgagaagacagtagcagctgtcacgaaggacaaggatgtgaacgctggttctcatgggaaaattgtgccgcgtctttcgaagatcacaaagaagatgtcactgccacgcgtgaaaggaaaagtcatactcgatatcgatcatttgctggaatataagccggatcaaattgagttgtataacacacgagcgtctcatcagcaattttcctcctggtttaatcaagttagaacagaatatgatttgaatgagcaacagatgggagttgtaatgaatggtttcatggtttggtgcattgaaaatggcacttcgcctgatatcaatggagtgtggttcatgatggatggagatgagcaggtcgagtatcctttgaaaccaatagtcgaaaatgcaaagccaacgctgcgacaaataatgcatcacttctcagatgcagcggaggcatacatagaaatgagaaatgcagaggcaccatacatgccgaggtatggtttgcttcgaaatctacgggataggagtttggctcgatacgctttcgacttctacgaagtcaactctaaaactcctgaaagagcccgcgaagctgttgcgcagatgaaagcagcagctcttagcaatgtttcttcaaggttgtttggccttgatggaaatgttgccaccactagcgaagacactgaacggcacactgcacgtgatgttaatagaaacatgcacaccctgttaggtgtgaacacaatgcagtaaagggtaggtcacctacctaggttatcgattcgctgccgacgtaattctaatatttaccgctttttatgatatctttagatttcagtgtgggcctcccacctttaaagcgtaaagtttatgttagttgtccaggaatgccgtagtcctgtcggaagctttagtgtgagcctctcacggataagctcgagattagactccgtttgcaagcct

SEQ ID NO: 10 - DNA, 37 bp, Artificial sequence
  Single strand DNA oligonucleotide
atggatccctgcagtcaggcactcagccaactgtggc

SEQ ID NO: 11 - DNA, 32 bp, Artificial sequence
  Single strand DNA oligonucleotide
atggcgccggtaccaggcttgcaaacggagtc

SEQ ID NO: 12 - DNA, 18 bp, Artificial sequence
  Single strand DNA oligonucleotide
gctacggagcgtccgcgg

SEQ ID NO: 13 - DNA, 20 bp, Artificial sequence
  Single strand DNA oligonucleotide
cgcggtcgactgtatgtcat

SEQ ID NO: 14 - DNA, 20 bp, Artificial sequence
  Single strand DNA oligonucleotide
tctgaccagactaccgaaaa

SEQ ID NO: 15 - DNA, 20 bp, Artificial sequence
  Single strand DNA oligonucleotide
atggcttacaatccgatcac

SEQ ID NO: 16 - DNA, 30 bp, Artificial sequence
  Single strand DNA oligonucleotide
gagaggatccatgtttctaagtcaggtcct

SEQ ID NO: 17 - DNA, 31 bp, Artificial sequence
  Single strand DNA oligonucleotide
gagagaattctcactttgaggaagtagcgct

SEQ ID NO: 18 - DNA, 18 bp, Artificial sequence
  Single strand DNA oligonucleotide
tctatcgcttaacgcagc

SEQ ID NO: 19 - DNA, 20 bp, Artificial sequence
  Single strand DNA oligonucleotide
atgtcttactctacttctgg

SEQ ID NO: 20 - DNA, 19 bp, Artificial sequence
  Single strand DNA oligonucleotide
caagacgaggtagacgaac

SEQ ID NO: 21 - DNA, 19 bp, Artificial sequence
  Single strand DNA oligonucleotide
atgccttactctaccagcg

(19) United States
(12) Patent Application Publication
(10) Pub. No.: US 2008/0016593 A1
(43) Pub. Date: Jan. 17, 2008

(54) Engrafted Plants Resistant To Viral Diseases And Methods Of Producing Same

(75) Inventors: Amit Gal-On, Ramat-Hasharon (IL); Aaron Zelcer, Rehovot (IL); Dalia Wolf, Ness Ziona (IL); Paul Vector Gaba, Rehovot (IL); Yehezkel Antignus, Moshav Neve Mivtach (IL)

Correspondence Address: FENNEMORE CRAIG, 3003 NORTH CENTRAL AVENUE, SUITE 2600, PHOENIX, AZ, 85012, US

(73) Assignee: STATE OF ISRAEL, MINISTRY OF AGRICULTURE, Bet Dagan (IL)

(21) Appl. No.: 10/590,376

(22) PCT Filed: Feb. 23, 2005
(86) PCT No.: PCT/IL05/00224

§ 371 Date: May 23, 2007

Related U.S. Application Data
(60) Provisional application No. 60/546,173, filed on Feb. 23, 2004.

(51) Int. Cl. A01H 5/00

(52) U.S. Cl. 800/279; 800/295

(57) ABSTRACT

The present invention relates to engrafted plants comprising a transgenic rootstock resistant to a viral disease and a susceptible scion, wherein the resistance to the disease is conferred to the scion from the transgenic viral-resistant rootstock, such that the entire engrafted plant is resistant to the viral disease. The invention also relates to methods of producing the engrafted viral-resistant plants and the plants produced thereof.

### FIELD OF THE INVENTION

The present invention relates to plants resistant to viral disease comprising a transgenic viral-resistant rootstock and an engrafted scion, in which the resistance to the disease is conferred to the scion from the transgenic viral-resistant rootstock, and to methods of producing same.

### BACKGROUND OF THE INVENTION

Plant pathogenic viruses cause significant losses in agricultural fresh produce all over the world. Modem agricultural practices, including the growth of a single species in wide regions, and the demand for fresh produce all year round leading to an increase in greenhouse area, aggravated the problem of viral spread and increase the resultant damage.

Traditional breeding programs for the production of plants resistant to viral infection have been used successfully in the past. However, such breeding programs depend on natural sources for resistance, which are not always available. For example, the Zucchini Yellow Mosaic Virus (ZYMV) causes severe damages in Cucurbitaceae every year all over the world. The virus is transferred from plant to plant by leaf aphids, and insecticides were found to be inefficient in preventing the virus spread. Moreover, limited sources of resistance have been identified.

Powell-Abel et al. (Powell-Abel et al., 1986. Science 232:738-743) were the first to show that plants transformed with and expressing the coat protein (CP) gene of tobacco mosaic virus (TMV) are resistant to TMV. Since then, viral coat protein-mediated resistance has been shown with at least 25 viruses in 15 taxonomic groups including alfalfa mosaic virus, tobacco rattle virus, potato virus X, cucumber mosaic virus (CMV), potyviruses, and plants transformed with both potato virus X and potato virus Y coat protein.

In general, CP-mediated resistance is effective against a wider range of viral strains, but is commonly less efficient than RNA-mediated resistance, i.e. wherein expression of viral RNA fragments that are not translated to protein impart viral resistance. For example, U.S. Pat. No. 6,649,813 discloses virus-induced resistance that may be transferred from one plant generation to another in which transgenic plants containing a coding sequence, taken from the read-through portion of the replicase portion of the viral genome, are resistant to subsequent disease by the virus. The use of the 54 kDa coding sequence from Tobacco Mosaic Virus (TMV) is specifically described. Replicase-mediated resistance is limited to strains that share high sequence homology, is not affected by the titer of the challenging virus, and is not correlated with the transgene expression level.

Posttranscriptional gene silencing (PTGS) is a sequence-specific defense mechanism that can target both cellular and viral mRNAs, and is a widely used tool for inactivating gene expression. PTGS is known to occur in plants, while a closely related phenomenon, RNA interference (RNAi), is known to occur in a wide range of other organisms (Baulcombe, D. 2000. Science, 290:1108-1109). RNA interference has been shown to occur, for example, in Caenorhabditis elegans, Neurospora crassa, Drosophila melanogaster and in mammals. In addition, transgenes and viruses have been shown to induce gene silencing in plants, and it is now believed that PTGS is a natural defense mechanism against virus accumulation (Hamilton A. and Baulcombe, D. 1999. Science 286:950-952; Matzke et al., 2001. Curr. Opin. Genet. Dev. 11:221-227).

Virus-induced gene silencing (VIGS) has been well demonstrated for a number of plant RNA viruses. The process is initiated by double-stranded RNA (dsRNA) molecules. The dsRNA molecules are possibly generated by replicative intermediates of viral RNAs or by aberrant transgene-coded RNAs, which become dsRNA by RNA-dependent RNA polymerase activity. The dsRNAs are cleaved by a member of the ribonuclease III family into short interfering RNAs (siRNAs), which generally range in size from 21 to 26 nucleotides. It is believed that the siRNAs then promote RNA degradation by forming a multi-component nuclease complex RISC (RNA Induced Silencing Complex) that destroys cognate mRNA. Since the discovery of siRNA, methods based on this mechanism have been utilized to silence specific target genes, as a research tool to elucidate the gene function and for the prevention of undesired gene expression.

WO 99/61631 discloses methods to alter the expression of a target gene in a plant using sense and antisense RNA fragments of the gene. The sense and antisense RNA fragments are capable of pairing and forming a double-stranded RNA molecule, thereby altering the expression of the gene.

WO 99/53050 discloses methods and means for reducing the phenotypic expression of a nucleic acid of interest in eukaryotic cells, particularly in plant cells, by introducing chimeric genes encoding sense and antisense RNA molecules directed towards the target nucleic acid, which are capable of forming a double stranded RNA region by base-pairing between the regions with the sense and antisense nucleotide sequence or by introducing the RNA molecules themselves.

WO 00/68374 relates to methods to alter the expression of a viral gene in a cell using sense and antisense RNA fragments of the gene. The sense and antisense RNA fragments are capable of pairing and forming a double-stranded RNA molecule, thereby altering the expression of the gene. The invention also relates to cells, plants or animals, obtained using the method of the invention, which are preferably resistant or tolerant to viruses.

WO 2004/009779 discloses compositions comprising precursor RNA constructs for the expression of an RNA precursor. The precursor RNA construct comprises a promoter that is expressed in a plant cell driving the expression of a precursor RNA having a microRNA. The miRNA is complementary or partially complementary to a portion of a target gene or nucleotide sequence and function to modulate expression of the target sequence or gene. In this manner, the RNA precursor construct can be designed to modulate expression of any nucleotide sequence of interest, either an endogenous plant gene or alternatively a transgene.

Grafting is an ancient technique used by farmers and gardeners to combine desired attributes of the rootstock with those of the scion. In the past, grafting was mainly used in perennials, specifically herbaceous plants and trees. Today, this technique is also used for annuals, and the percentage of engrafted vegetable seedlings comprising a rootstock and a scion increases constantly. Smirnov et al. (Smirnov et al., 1997. Plant Physiol. 114:1113-1121) used the grafting technique to engraft wild-type tobacco plants on transgenic tobacco plants expressing the pokeweed anti-viral protein. They demonstrated that expression of the anti-viral protein in the transgenic rootstock of the engrafted plants induces resistance to viral infection in the wild-type scion. However, resistance was depended on the enzymatic activity of the pokeweed anti-viral protein.

Hitherto, attempts to produce plants resistant to pathogen infection concentrated mainly on employing transformation methods to incorporate resistance-related trait into the plant genome. However, agricultural products obtained from transgenic plants are not desired in many countries. Alternatively, the grafting technique was used. Although use of a rootstock resistant to a certain disease has been shown, the engrafted scion was susceptible to transmission of the pathogen.

Thus, there is a recognized need for, and it would be highly advantageous to have plants resistant to pathogens, specifically to viruses, wherein the agricultural product is produced by a plant part which is not genetically modified.

### SUMMARY OF THE INVENTION

The present invention provides engrafted plants comprising a transgenic viral-resistant rootstock and a susceptible scion, wherein the entire plant is resistant to viral disease. The plants of the present invention can be perennial or annual. The present invention further provides compositions and methods for the production of the engrafted resistant plants. The nature of viral resistance depends on the specific features of the composition employed to produce a particular plant. According to certain aspects, the present invention provides engrafted plants protected from soil-borne viruses. According to other aspects, the plants of the present invention are resistant to foliage infection caused by a virus.

The plants of the present invention can be produced by various type of grafting; the method of grafting is typically used when the scion produces the desired agricultural product. Advantageously, the agricultural product produced by the plants of the present invention is not genetically modified, as the rootstock is the only transgenic part of the plant.

Without wishing to be bound to any particular theory or mechanism, the resistance of the plants of the present invention to viral disease may be attributed to RNA-mediated viral resistance imparted to a transgenic rootstock, which confers resistance to an engrafted susceptible scion.

Thus, according to one aspect, the present invention provides a plant comprising a transgenic rootstock resistant to viral disease other than by means of expression of an anti-viral protein, and a scion susceptible to the viral disease, wherein the engrafted plant is resistant to said viral disease.

According to various embodiments, the present invention provides viral resistance conferred to the engrafted scion by a transgenic rootstock expressing a nucleic acid sequence transcript selected from a sequence encoding a viral protein or part thereof and an siRNA. Thus, according to certain embodiments, the transgenic rootstock resistant to viral infection comprises a nucleic acid sequence having at least 90% identity to at least one segment of the viral genome. According to additional embodiment, the transgenic rootstock resistant to viral infection comprises a DNA construct designed for generating siRNAs targeted to at least one segment of the viral genome.

As used herein, the term “segment” refers to a nucleic acid sequence selected from the group consisting of a coding region of the viral genome, a non-coding region, parts thereof and combinations of same.

According to one embodiment, the nucleic acid sequence having at least 90% identity to at least one segment of the viral genome encodes a protein or part thereof. According to another embodiment, the nucleic acid sequence encodes a protein selected from the group consisting of a coat protein, a replication protein, a movement protein or parts thereof. According to one embodiment, the transgenic rootstock comprises a nucleic acid sequence being a segment of the replicase portion of the virus genome.

According to another embodiment, the transgenic rootstock comprises a nucleic acid sequence encoding a putative 54 kDa protein being a fragment of the replication protein of cucumber fruit mottle mosaic virus (CFMMV). According to one currently preferred embodiment, the transgenic rootstock comprises a nucleic acid sequence having the sequence set forth in SEQ ID NO:1.

According to yet another embodiment, the DNA construct designed for generating siRNAs targeted to at least one segment of a viral genome comprises:

(a) at least one plant expressible promoter operably linked to;

(b) a nucleic acid sequence encoding an RNA sequence that forms at least one double stranded RNA, wherein the double stranded RNA molecule comprises a first nucleotide sequence of at least 20 contiguous nucleotides having at least 90% sequence identity to the sense nucleotide sequence of the target segment of the viral genome and a second nucleotide sequence of at least 20 contiguous nucleotides having at least 90% sequence identity to the complementary sequence of the sense nucleotide sequence of said target segment of said viral genome; and optionally

(c) a transcription termination signal.

According to one preferred embodiment, the DNA construct designed for generating siRNAs targeted to at least one segment of a viral genome comprises:

(a) at least one plant expressible promoter operably linked to;

(b) a nucleic acid sequence encoding an RNA sequence that forms at least one double stranded RNA in the form of stem-loop, wherein the double stranded RNA molecule comprises a first nucleotide sequence of at least 20 contiguous nucleotides having at least 90% sequence identity to the sense nucleotide sequence of the target segment of the viral genome; a second nucleotide sequence of at least 20 contiguous nucleotides having at least 90% identity to the complementary sequence of the sense nucleotide sequence of said target segment of said viral genome; and a spacer sequence; and optionally,

(c) a transcription termination signal.

It is to be understood that the practice of the present invention is not limited to any specific DNA construct, providing the construct is designed to direct the generation of siRNAs within the plant cell, wherein the siRNAs are targeted to at least one segment of a viral genome. According to certain embodiments, the construct comprises nucleic acid sequence encoding an RNA sequence that forms at least one double-stranded RNA molecule, wherein the double stranded RNA molecule mediates cleavage of the viral target sequence. The DNA construct may be designed to form double stranded RNA in various ways. Moreover, it should be understood that although the present invention is practiced with a construct generating siRNAs, any method known in the art for the generation of siRNAs within a plant cell is also encompassed within the scope of the present invention.

The use of siRNA as a mean to cleave a segment of a viral genome within a plant cell has been previously disclosed. It has been also shown that when a chromosomal gene (whether endogenous or heterologous gene) is silenced in a rootstock, silencing is transmitted from the silenced rootstock to a target scion expressing the corresponding chromosomal gene. However, the present invention discloses that surprisingly, transforming a rootstock with a construct generating siRNAs targeted to at least a segment of the genome of a pathogenic virus, confers resistance to a grafted scion, which is otherwise susceptible to infection by the virus.

According to some embodiments, the first and the second nucleotide sequences are operably linked to the same promoter. In other embodiments, each of the first and the second nucleotide sequences is operably linked to a separate promoter, wherein the separate promoters may be the same or different.

According to one embodiment, the first nucleotide sequence includes a sequence of at least 20 contiguous nucleotides which are at least 95% identical, preferably 100% identical to the sequence of the sense nucleotide sequence of the at least one segment of the viral genome. According to another embodiment, the second nucleotide sequence includes a sequence of at least 20 contiguous nucleotides which are at least 95% identical, preferably 100% identical to the sequence of the complement of the sense nucleotide sequence of at least one segment of the viral genome.

There is no upper limit to the length of the first and the second nucleotide sequences that can be used, such that the construct of the present invention can include nucleotide sequences of varying lengths, including those from about 20 nucleotides to the full length of the target RNA. Preferably, the length of the first and the second nucleotide according to the present invention is about 1,000 nucleotides in length. According to another embodiment, the length of the first and the second nucleotide is about 22 nucleotides in length.

According to one preferred embodiment, the first nucleotide sequence comprises a nucleotide sequence having 90% identity, preferably 95%, more preferably 100% identity to the nucleotide sequence set forth in SEQ ID NO:2 or a fragment thereof. According to another currently preferred embodiment, the second nucleotide sequence comprises a nucleotide sequence having 90% identity, preferably 95%, more preferably 100% identity to the complement of the nucleotide sequence set forth in SEQ ID NO:2 or a fragment thereof.

According to one embodiment, the structure of the inhibitory RNA molecule comprises further to the first and the second nucleotide sequences a spacer sequence, thus the double stranded RNA is in a form of stem-loop RNA (hairpin RNA, hpRNA). In a preferred embodiment, the length of the spacer sequence is ⅕ to 1/10 of the length of the first and the second nucleotides.

According to certain embodiments, the spacer comprises a nucleotide sequence derived from a gene intron to enhance siRNA production. According to one embodiment, the spacer comprises a nucleotide sequence comprising an intron from the castor bean catalase gene, having the sequence set forth in SEQ ID NO:3.

Optionally, the construct encoding the siRNA comprises a transcription termination signal. According to one embodiment, the transcription termination signal is the NOS terminator.

According to certain embodiments, the nucleic acid sequence conferring resistance to the engrafted scion, further comprises regulatory elements for the expression of the nucleic acid sequence within a plant cell. The expression control elements are selected from the group consisting of a promoter, an enhancer, a transcription factor, a splicing signal, and a termination sequence. According to one embodiment, the promoter is a constitutive promoter. According to one currently preferred embodiment, the constitutive promoter is the promoter of strawberry vein banding virus. According to another embodiment, the promoter is tissue specific promoter.

According to other embodiments, the transgenic rootstock transformed with a nucleic acid sequence having at least 90% identity to at least one segment of the viral genome is resistant to a disease caused by a soil-borne virus. According to one embodiment, the engrafted plant comprising such transgenic rootstock is protected from a soil-borne virus selected from the group consisting of nematode-transmitted viruses, fungal-transmitted viruses, viruses transmitted via root wound and viruses transmitted via unknown vector.

According to one embodiment, nematode-transmitted viruses are selected from, but not limited to, Nepoviruses: Arabis mosaic virus, Grapevine fanleaf virus, Tomato black ring virus, Raspberry ringspot virus, Tomato ringspot virus, and Tobacco ringspot virus; Tobraviruses: Pea early browning virus, Tobacco rattle virus and Pepper ringspot virus.

According to another embodiment, fungal-transmitted viruses are selected from the group consisting of, but not limited to, Cucumber leafspot virus, Cucumber necrosis virus, Melon necrotic spot virus, Red clover necrotic mosaic virus, Squash necrosis virus, Tobacco necrosis satellite virus, Lettuce big-vein virus, Pepper yellow vein virus, Beet necrotic yellow vein virus, Beet soil-borne virus, Oat golden stripe virus, Peanut clump virus, Potato mop top virus, Rice stripe necrosis virus, Soil-borne wheat mosaic virus, Barley mild mosaic virus, Barley yellow mosaic virus, Oat mosaic virus, Rice necrosis mosaic virus, Wheat spindle streak mosaic virus and Wheat yellow mosaic virus.

According to further embodiment, viruses transmitted via root wound are selected from the group consisting of, but not limited to, Tobamovirus genera: Tobacco mosaic virus, Tomato mosaic virus, Cucumber green mottle mosaic tobamovirus, Cucumber fruit mottle mosaic virus, Kyuri green mottle mosaic virus, Odontoglossum ringspot virus, Paprika mild mottle virus, Pepper mild mottle virus, Ribgrass mosaic virus and Tobacco mild green mosaic virus.

According to yet another embodiment, viruses transmitted by unknown rout are selected from the group consisting of, but not limited to, Watercress yellow spot virus, Broad been necrotic wilt virus, Peach rosette mosaic virus and Sugarcane chlorotic streak virus.

According to one embodiment, the engrafted plant is protected from a disease caused by a soil-borne virus of the tobamovirus genus. According to another embodiment, the engrafted plant is protected from a disease caused by the tobamovirus CFMMV. According to yet another embodiment, the engrafted plant is selected from the Cucurbitaceae family.

The present invention shows for the first time that it is possible to impart resistance to soil-borne viral pathogens to susceptible plants, using the grafting technique. Grafting a susceptible scion with a resistant rootstock, wherein the resistant rootstock comprises a nucleic acid sequence having at least 90% identity to at least one segment of the soil-borne viral genome, reverse the scion from being susceptible to being protected towards the soil-borne pathogen.

According to additional embodiments, engrafted plants comprising a transgenic viral-resistant rootstock comprising a DNA construct designed to generate siRNAs targeted to at least one segment of the viral genome and a scion, encompass plants of any species. Furthermore, a plant can be produced to exhibit resistance to any selected plant virus, wherein resistance to a plurality of plant viruses can be also obtained. According to certain embodiments, the plant is resistant to a soil-borne virus selected from the group described herein above. According to other embodiments, the plant is resistant to a virus transmitted by a vector affecting the aerial part of the plant. According to one embodiment, the virus affecting the aerial part of the plant is of a virus family selected from the group consisting of Caulimoviridae, Geminiviridae, Circoviridae, Reoviridae, Tartitiviridae, Bromoviridae, Comoviridae, Potyviridae, Tombusviridae, Sequiviridae, Clostroviridae and Luteoviridae. According to another embodiment, the virus is selected from the group consisting of Tobamovirus, Tobravirus, Potexvirus, Carlavirus, Allexivirus, Capillovirus, Foveavirus, Trichovirus, Vitivirus, Furovirus, Pecluvirus, Pomovirus, Benyvirus, Hordeivirus, Sobemovirus, Marafivirus, Tymovirus, Idaeovirus, Ourmivirus, and Umbravirus.

According to certain embodiments, the engrafted plants comprising a rootstock comprising a DNA construct designed for generating siRNAs are resistant to a plant virus from the Potyviridae family. In the description that follows, the use of siRNA targeted to the 3′ end of Zucchini Yellow Mosaic Virus (ZYMV) genome, including the coat protein gene and the 3′ non coding region impart resistance to the virus on transgenic Nicotiana benthamiana rootstock and further to N. benthamiana scion is described as a specific example of the broader technology according to the present invention.

The nucleic acid sequences transformed to the rootstocks of the present invention preferably further comprise a selectable marker, such that only transgenic plants can germinate and develop. Additionally or alternatively, a reporter gene can be incorporated into the construct, as to enable selection of transgenic plants expressing the reporter gene. According to one embodiment, the selection marker is a gene inducing antibiotic resistance within the plant.

Recent regulations drawn with regard to the growth of transgenic plants in open fields preclude the use of transgenic plants comprising a gene inducing antibiotic resistance. Thus, selection of transgenic plants can be performed by co-transformation of a first construct designed to confer viral resistance according to the present invention and a second construct comprising a reporter gene. Successful transformation of the construct conferring viral resistance is then verified by methods known to a person skilled in the art, for example by PCR, only in plants expressing the reporter gene and thus indicating a successful transformation.

The nucleic acid sequence of the present invention may be incorporated into a plant transformation vector used to transform plants, as is known in the art. According to additional aspect, the present invention provides a method for producing a plant resistant to infection by a virus comprising the steps of (a) providing a transgenic rootstock resistant to the viral infection other than by means of expression of an anti-viral protein; (b) providing a scion susceptible to infection by said virus; and (c) grafting the scion onto the rootstock as to obtain an engrafted plant resistant to said viral infection.

According to one embodiment, the rootstock is transformed with a nucleic acid sequence having at least 90% identity to at least one segment of a viral genome as to produce a transgenic rootstock resistant to the viral infection. According to another embodiment, the rootstock is transformed with a DNA construct designed for generating siRNAs targeted to at least one segment of a viral genome as to produce a transgenic rootstock resistant to the viral infection. According to preferred embodiments of the invention, the affected segment of the viral genome is essential for the virus for plant infection and/or replication, so that its cleavage prevents the viral infection and/or replication, thereby providing a resistant plant.

Transformation of plants with a polynucleotide or a DNA construct to produce resistant rootstocks may be performed by various means, as is known to one skilled in the art. Common methods are exemplified by, but are not restricted to, Agrobacterium-mediated transformation, microprojectile bombardment, pollen mediated transfer, liposome mediated transformation, direct gene transfer (e.g. by microinjection) and electroporation of embryogenic calli. According to one embodiment, resistant plants are produced using Agrobacterium mediated transformation.

Transgenic plants comprising the nucleic acid sequences of the present invention may be selected employing standard methods of molecular genetic, known to a person of ordinary skill in the art. According to one embodiment, the transgenic plants are selected according to their resistance to antibiotic. According to certain embodiments, the antibiotic serving as a selectable marker is one of the aminoglycoside group consisting of paromomycin and kanamycin.

According to another embodiment, the transgenic plants are selected according to their resistance to the viral infection. According to one embodiment, the transgenic plants are selected according to their resistant to a soil-borne virus selected from the group consisting of, but not limited to, nematode-transmitted viruses: Nepoviruses: Arabis mosaic virus, Grapevine fanleaf virus, Tomato black ring virus, Raspberry ringspot virus, Tomato ringspot virus, and Tobacco ringspot virus; Tobraviruses: Pea early browning virus, Tobacco rattle virus and Pepper ringspot virus; fungal-transmitted viruses: Cucumber leafspot virus, Cucumber necrosis virus, Melon necrotic spot virus, Red clover necrotic mosaic virus, Squash necrosis virus, Tobacco necrosis satellite virus, Lettuce big-vein virus, Pepper yellow vein virus, Beet necrotic yellow vein virus, Beet soil-borne virus, Oat golden stripe virus, Peanut clump virus, Potato mop top virus, Rice stripe necrosis virus, Soil-borne wheat mosaic virus, Barley mild mosaic virus, Barley yellow mosaic virus, Oat mosaic virus, Rice necrosis mosaic virus, Wheat spindle streak mosaic virus and Wheat yellow mosaic virus; viruses transmitted via root wound: Tobamovirus genera: Tobacco mosaic virus, Tomato mosaic virus, Cucumber green mottle mosaic tobamovirus, Cucumber fruit mottle mosaic virus, Kyuri green mottle mosaic virus, Odontoglossum ringspot virus, Paprika mild mottle virus, Pepper mild mottle virus, Ribgrass mosaic virus and Tobacco mild green mosaic virus; and viruses transmitted by unknown rout: Watercress yellow spot virus, Broad been necrotic wilt virus, Peach rosette mosaic virus and Sugarcane chlorotic streak virus.

According to another embodiment, the transgenic plants are selected according to their resistant to a virus transmitted by a vector affecting the aerial part of the plant, selected from the group consisting of a virus family: Caulinioviridae, Geminiviridae, Circoviridae, Reoviridae, Tartitiviridae, Bromoviridae, Comoviridae, Potyviridae, Tombusviridae, Sequiviridae, Clostroviridae and Luteoviridae; Tobamovirus, Tobravirus, Potexvirus, Carlavirus, Allexivirus, Capillovirus, Foveavirus, Trichovirus, Vitivirus, Furovirus, Pecluvirus, Pomovirus, Benyvirus, Hordeivirus, Sobemovirus, Marafivirus, Tynmovirus, Idaeovirus, Ourmivirus, Umbravirus.

According to another aspect the present invention relates to engrafted plants generated by the methods of the present invention. The plants, comprising a scion which is otherwise susceptible to viral infection grafted onto a transgenic, viral-resistant rootstock, are resistant to the viral infection. The engrafted plant would be resistant to the same virus species to which the rootstock is resistant. The rootstock comprises a nucleic acid sequence according to the invention stably integrated into its genome, wherein the nature of the DNA construct determines the virus species to which it is resistant.

These and additional features of the present invention are explained in greater detail in the figures, description and claims below.

### BRIEF DESCRIPTION OF THE FIGURES

FIG. 1 shows a schematic representation of the CFMMV genome organization and of the 54-kDa DNA construct. A. Organization of the CFMMV genome. The nucleotide numbers refer to the positions of the putative genes. The three sub-genomic RNAs encoding the putative 54-kDa (RNA-II), the movement protein (MP) and coat protein (CP) genes are marked. B. Composition of the construct used in plant transformation, between the left (LB) and the right (RB) T-DNA borders. The 54-kDa gene of CFMMV was fused to the ZYMV 5′ non-coding region (NCR) between the truncated SVBV (SV) promoter and the NOS poly-A terminator (T). The selective NPTII gene was inserted under control of the full-length SVBV promoter.

FIG. 2 shows a schematic representation of the DNA construct for generating siRNAs, pCddCP-ZY between the left (LB) and the right (RB) T-DNA borders. Inverted repeats of a polynucleotide comprising the ZYMV gene coat protein (CP) and the 3′non-coding region (NCR) of the virus were fused from each side of an intron, between the SVBV (SV) promoter and the NOS poly-A terminator (Ter). The selective NPTII and GUS gene was inserted each under control of 35S promoter

FIG. 3 demonstrates the response of 144 resistant plants and control plants to infection with different cucurbit-infecting tobamoviruses; CFMMV (CF), CGMMV (CG), KGMMV (KG) and ZGMMV (ZG), as assessed by virion accumulation (a) and RT-PCR (b).

FIG. 4 shows RT-PCR products indicating infection by CFMMV. Total RNA from line I44 and non-transformed ‘Ilan’ plants, either non-inoculated (H) or three weeks after inoculation with CFMMV (inocul.), served as a template for RT-PCR (a) and (b) and PCR (c) as a negative control analysis. (a) primers for the 54-kDa gene. (b) primers for the CP gene. MW: molecular weight of 100-bp stepladder.

FIG. 5 shows the response of cucumber plants to CFMMV inoculation. A and C: the transgenic line I44. B and D: non-transformed ‘Ilan’ cultivar. Leave and fruit displayed disease symptoms three and seven weeks after inoculation, respectively.

FIG. 6 shows the presence of 54-kDa transcripts in transformed I44 plants. Total RNA was extracted from the second and third true leaves of transformed (144) and non-transformed plants (‘Ilan’), either non-inoculated or 18 dpi with CFMMV (CF) or with ZYMV (ZY). RNA was analyzed by denaturing agarose gel electrophoresis and Northern blot hybridization. The amount of RNA loaded per lane was either 3 μg (from non-transformed ‘Ilan’ plants infected with CFMMV), or 30 μg (all other lanes). A ³²P-labeled RNA probe complementary to the 54-kDa coding sequence was used for detecting the transgene as well as viral RNA. The electrophoretic positions of CFMMV RNA and subgenomic RNA-I1 are indicated adjacent to the panels. The transcript of the transgene is marked (54-kDa transcript). Lane Ilan+CF (non-transformed plant infected with CFMMV) was exposed for 4 h, whereas the other lanes were exposed for 3 days. Bottom panel: 18S RNA level as determined by ethidium, bromide staining of the gel prior to transfer to the membrane.

### DETAILED DESCRIPTION OF THE INVENTION

The present invention relates to engrafted plants resistant to viral disease, comprising a rootstock and a scion. Plants transformed with a DNA construct conferring resistance to a plant virus other than via the expression of anti-viral protein serve as a source for rootstocks, and plants susceptible to the viral disease serve as a source for scions. Upon grafting the scion onto the rootstock the entire plant is protected from the viral infection.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

### Definitions

The term “plant” is used herein in its broadest sense. It includes, but is not limited to, any species of woody, herbaceous, perennial or annual plant. It also refers to a plurality of plant cells that are largely differentiated into a structure that is present at any stage of a plant's development. Such structures include, but are not limited to, a root, stem, shoot, leaf, flower, petal, fruit, etc.

As used herein, the term “engrafted plant” refers to a plant comprising a rootstock and a scion, wherein the scion is grafted onto the rootstock by any method known in the art.

As used herein, the term “rootstock” refers to a stock for grafting comprising the root part of a plant. The term “scion” refers to a detached living portion of a plant designed or prepared for union with a stock in grafting, usually supplying solely or predominantly aerial parts to the graft.

As used herein, the term “virus” refers to a plant virus, i.e. a virus capable of infecting a plant cell and propagating within the plant cell. Typically, the virus is pathogenic, such that substantial viral infection causes yield lose in agricultural crops.

As used herein, the term “soil-borne” virus refers to viruses that are transmitted by soil vectors including nematodes, fungi or unknown soil vectors, and viruses which remain in plant debris and are transmitted in the soil via a root wound.

The terms “resistant plant” and “plant resistant to viral disease” refer to a plant having an increased tolerance to the virus compared to a non-resistant (susceptible) plant. The increased tolerance is examined by deliberate infection of the plant with the virus in question. Plants showing lower symptom intensity compared to susceptible plant, according to a symptom scale specific for each virus, are defined as plants resistant to the virus. Plants can either resist infection (resistance is then referred to as immunity) or undergo a preliminary phase of infection from which they recover (resistance is then referred to as recovery). Resistance can be a stable trait, which can be inherited to the offspring population. Alternatively, resistance exists only as long as the engrafted plant comprises a rootstock and a scion. In the later situation, a plant resistant to a viral disease is also referred to as a plant protected from the viral disease.

The term “gene” refers to a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. As used herein, the term “intron”refers to a non-coding sequence interrupting the coding region of a gene.”Introns are removed or “spliced out” from the nuclear or primary transcript, and therefore are absent in the messenger RNA (mRNA) transcript.

The term “nucleic acid” as used herein refers to RNA or DNA that is linear or branched, single or double stranded, or a hybrid thereof. The term also encompasses RNA/DNA hybrids.

The terms “promoter element,” “promoter,” or “promoter sequence” as used herein, refer to a DNA sequence that is located at the 5′ end (i.e. precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

The terms “heterologous gene” or “chimeric genes” refers to a gene encoding a factor that is not in its natural environment (i.e., has been altered by the hand of man). For example, a heterologous gene includes a gene from one species introduced into another species. A heterologous gene also includes a gene native to an organism that has been altered in some way (e. g., mutated, added in multiple copies, linked to a non-native promoter or enhancer sequence, etc.). Heterologous genes may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript). Heterologous plant genes are distinguished from endogenous plant genes in that the heterologous gene sequences are typically joined to nucleotide sequences comprising regulatory elements such as promoters that are not found naturally associated with the gene for the protein encoded by the heterologous gene or with plant gene sequences in the chromosome, or are associated with portions of the chromosome not found in nature (e.g., genes expressed in loci where the gene is not normally expressed). A plant gene endogenous to a particular plant species (endogenous plant gene) is a gene which is naturally found in that plant species or which can be introduced in that plant species by conventional breeding.

The term “transgenic” when used in reference to a plant or fruit or seed (i.e., a “transgenic plant” or “transgenic fruit” or a “transgenic seed”) refers to a plant or fruit or seed that contains at least one heterologous gene in one or more of its cells. The term “transgenic plant material” refers broadly to a plant, a plant structure, a plant tissue, a plant seed or a plant cell that contains at least one heterologous gene in at least one of its cells.

The terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

The present invention discloses a system utilizing the technique of grafting to produce transgenic plants resistant to viral infection, wherein the rootstock is the only genetically modified part of the plant. Thus, the plants of the present invention are advantageous over hitherto known resistant plants in that transformation methods can be used to impart resistant to viral infection, while the agricultural products produced by the plants are not genetically modified.

The grafting of a scion upon a rootstock is a common horticultural practice used for many years in the propagation of woody plants. Once grafted, water and nutrients are transported from the rootstock to the scion to support growth of the scion. As of today, grafting is widely used with a variety of plants species, to improve the horticultural traits of the resulted grafted plant. The percentage of engrafted seedling comprising a rootstock and scion used in field crops is growing constantly in modern agriculture practice. For example, about 60% of cucumber and watermelon seedlings grown in Greece, Italy and Spain are engrafted seedlings. To meet the growing demand for grafted seedlings, various methods have been developed for high throughput grafting. In all methods employed, complementary ends of the scion and the rootstock are brought together to form a graft union. Callous tissue forms at the graft union as part of the normal healing process of the plant and serves as a conduit for water and nutrients between the scion and rootstock.

According to one aspect, the present invention provides a plant comprising a transgenic rootstock resistant to viral disease other than by means of expression of an anti-viral protein and a scion susceptible to the viral disease, wherein the engrafted plant is resistant to said viral disease.

Various methods may be employed to produce transgenic plants resistant to viral disease that may serve as rootstocks. The present invention relates specifically to transgenic rootstocks expressing transcripts homologous to segments of the target virus genome.

Without wishing to be bound to a specific mechanism, the resistance may be associated with RNA silencing. RNA silencing, termed post-transcriptional gene silencing (PTGS) in plants, quelling in fungi, and RNA interference in animals, refers to the phenomenon whereby specific gene transcript levels are reduced in the presence of related RNA. The silenced gene may be endogenous or exogenous to the organism, present integrated into a chromosome or present in a transient form, such as transfection vector or virus that is not integrated into the genome. The expression of the gene is either completely or partially inhibited. PTGS may also be considered to inhibit the function of a target RNA, completely or partially.

According to certain embodiments the transgenic rootstock resistant to viral infection comprises a nucleic acid sequence having at least 90% identity to at least one segment of the viral genome.

It has been shown that introducing a transgene constitutively expressing part of the genome of a virus lead to resistance of the plant to infection by the virus (Marathe et al., 2000. Plant Mol. Biol. 43:295-306). The present invention now shows that grafting a susceptible scion on a transgenic rootstock transformed as described above, results in imparting the viral resistance from the rootstock to the scion. Particularly, the present invention shows that such engrafted plant is protected from disease caused by soil-borne viruses.

As a non-limiting example, the present invention discloses the protection of a susceptible cucumber cultivar against the soil-borne cucumber fruit mosaic tobamovirus (CFMMV) by grafting onto a transgenic resistant cucumber rootstock.

CFMMV is a newly reported cucurbit-infecting tobamovirus isolated from greenhouse-grown cucumbers (Cucumis sativus L.) in Israel (Antignus et al., 2001. Phytopathology 91:565-571). Cucumber varieties are susceptible to four distinct tobamoviruses that belong to two subgroups. CFMMV is closely related biologically and sequence-wise to Kyuri green mottle mosaic virus (KGMMV) and zucchini green mottle mosaic virus (ZGMMV), but displays a weaker serological affinity and lower coat protein (CP) homology with cucumber green mottle mosaic virus (CGMMV-W).

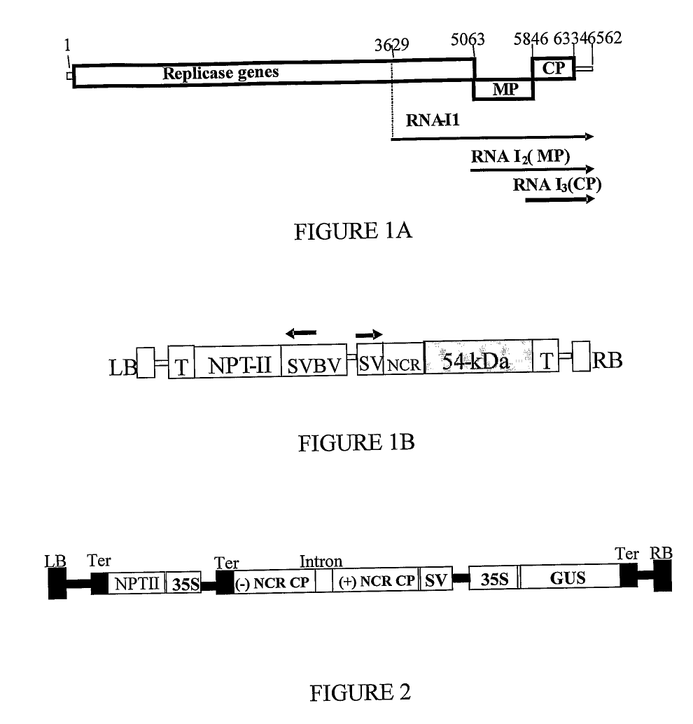

The CFMMV RNA genome consists of 6,562 nucleotides (Genebank accession no. AF321057, SEQ ID NO:4) with three subgenomic RNAs, coding four open reading frames (FIG. 1). The 5′ proximal region of CFMMV encodes two co-initiated proteins essential for replication: the 132-kDa and 189-kDa proteins. The 189-kDa protein is created by read-through of a leaky UAG terminator codon at the 3′-end of the 132-kDa protein at position 3629 (Antignus et al., 2001, supra). In tobacco mosaic virus (TMV) a subgenomic RNA termed II-RNA starts from the end of the short replicase gene (132-kDa) and encodes the read-through portion of the replicase frame, resulting in a putative 54-kDa protein, not yet identified in plants infected by tobamoviruses (Zaitlin, M. 1999. Phil Trans R Soc Lond B 354: 587-591).

Under commercial greenhouse conditions, symptoms of CFMMV are first recognized on fruits and apical leaves at a relatively advanced growth stage. Leaf symptoms include severe mosaic, vein banding and yellow mottling. In some cases, fully developed plants show severe wilting symptoms that lead to plant collapse. Rapid viral spread within greenhouses may lead to significant economic crop losses. The virus spreads easily via mechanical contact of plant organs with a source of inoculum. The virus can persist for long periods in plant residues or in infested greenhouse soil. The lack of efficient natural resistance sources in cucumber precludes the introgression of resistance into commercial varieties by traditional breeding programs.

Thus, according to one embodiment, the transgenic rootstock comprises a nucleic acid sequence encoding a putative 54 kDa protein being a fragment of the replication protein of cucumber fruit mottle mosaic virus (CFMMV). According to one currently preferred embodiment, the transgenic rootstock comprises a nucleic acid sequence having the sequence set forth in SEQ ID NO:1.

As exemplified herein below, parthenocarpic cucumbers transformed with the putative non-structural 54-kDa gene of CFMMV exhibited a high level of resistance (immunity) to CFMMV infection, and no traces of virus could be detected in inoculated plants by biological or molecular methods.

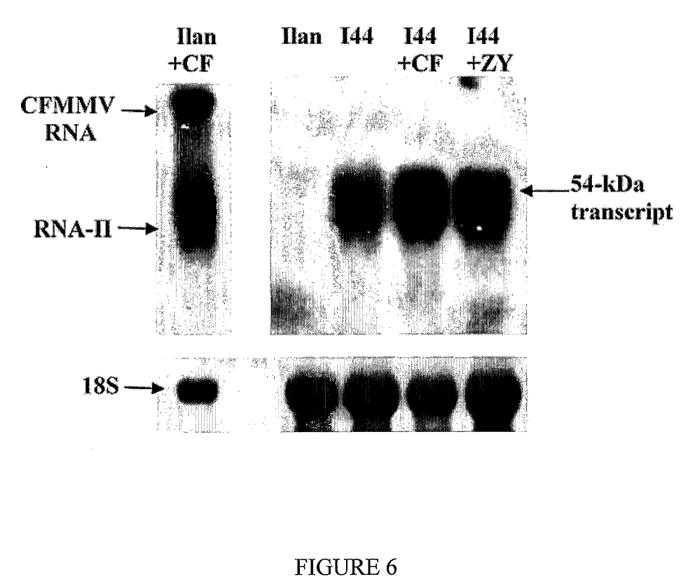

Several parameters associated with the resistance response were examined. In repeated experiments, the accumulation level of the 54-kDa locus transcript in the transgenic line was consistently low (FIG. 6), in spite of being driven by a strong constitutive promoter. Without wishing to be bound to a specific mechanism, resistance may be related to specific degradation of the transgene transcript, as was previously reported for silencing-mediated resistance to plant viruses.

Interestingly, inoculation of the transgenic plants with CFMMV or ZYMV did not affect the RNA expression level of the 54-kDa coding sequence (FIG. 6). In contrast, in several studies where silencing was implied in the resistance mechanism, inoculation of the transgenic resistant plants with the homologous virus reduced the level of the viral RNA (Savenkov, E. I. and Valkonen, J. P. 2002. J Gen Virol 83:2325-2335). Prior infection with viruses known to harbor a silencing-suppressing gene did not break the resistance of the transgenic plants to challenging CFMMV infection. This observation differs from the reports on the silencing-mediated resistance of transgenic N. benthamiana to Potato virus A or Plum pox virus, which was overcome by pre-infection with Potato virus Y and, respectively.

The present invention shows protection of susceptible cucumbers against soil inoculation with CFMMV, by grafting on a transgenic, resistant rootstock. Thus, the present invention demonstrates for the first time, that susceptible scions can be protected against soil borne viruses, by grafting on a transgenic rootstock expressing a transcript homologous to a segment of the virus genome. The transgenic-rootstock mediated protection disclosed herein has significant agricultural applications, enabling non-genetically modified (non-GMO) produce to be grown and adequately protected against soil pathogens.

According to additional embodiments, the transgenic rootstock resistant to viral infection comprises a DNA construct designed for generating siRNAs targeted to at least one segment of a viral genome.

The phenomenon of post-transcriptional gene silencing can be triggered by two types of transgene loci. The first type corresponds to highly transcribed single transgene, as described herein above. The second type of transgene loci that efficiently triggers PTGS is those carrying two transgene copies arranged as an inverted repeat (IR) producing dsRNA by read-through transcription. Double-stranded RNA (dsRNA) is remarkably effective at suppressing specific gene expression in a number of organisms, including plants. Virus-induced gene silencing (VIGS), for example, has been demonstrated for a number of plant RNA viruses (Vance and Vaucheret, 2001. Science 292:2277-2280). The process is initiated by double-stranded RNA (dsRNA) molecules. The dsRNA molecules are possibly generated by replicative intermediates of viral RNAs or by aberrant transgene-coded RNAs, which become dsRNA by RNA-dependent RNA polymerase activity (Dalmay et al., 2000. Cell 101:543-553; Waterhouse et al., 2001. Nature 411:834-842). Such dsRNA molecules have been incorporated into plants cells, and shown to be useful in suppressing or inhibiting viral gene expression (see, for example, US Application No. 20020169298).

Within the plant cell, the dsRNAs are cleaved by a member of the ribonuclease III family into short interfering RNAs (siRNAs), which generally range in size from 21 to 26 nucleotides. It is believed that the siRNAs then promote RNA degradation by forming a multi-component nuclease complex RISC (RNA Induced Silencing Complex) that destroys cognate mRNA (Elbashir et al., 2001. EMBO J 20:6877-6888; Zamore et al., 2000. Cell 101:25-33). Recently, it has been shown that such siRNAs ranging in size from 21 to 26 nucleotides, an intermediate of the RNAi pathway, are equally effective in suppressing gene expression in animal and mammalian systems. The use of siRNAs, therefore, has become a powerful tool for down regulating gene expression.

Posttranscriptional gene silencing spreads systemically throughout the individual plants in a very characteristic manner reminiscent of viral spread. This has led to the hypothesis of a systemic silencing signal that is produced in the tissues where silencing is initiated and is then transmitted to the distant parts of the plant where it can initiate silencing in a sequence-specific manner. The sequence specificity of the silencing suggest that the signal is a nucleic acid, but the identity of the signal remains unknown (Kalantidis, K. 2004. PloS Biology 2:1059-1061). Silencing spreads mainly in the direction from carbon source to carbon sink, that is, from tissues such as leaves that export the sugar products of photosynthesis, to tissues such as roots that import these products, and it can take up to several weeks until it is established in the whole plant. The existence of silencing signal has been shown in engrafted plants, whereby silencing was transmitted from silenced rootstock to target scion. However, such signal transmission depended on the expression of the corresponding transgene by the scion. Moreover, It was demonstrated that over-expression of a chromosomal corresponding gene, whether endogenous gene or stably integrated exogenous gene in the scion is an essential pre-requisite for triggering RNA degradation mediated by signal transmission from rootstock to scion.

The present invention demonstrates that transgenic rootstock expressing dsRNA targeted to silence a viral genomic sequence confers viral resistance to a susceptible scion upon grafting. The viral resistance imparted to the scion implies that the signal transferred from the rootstock to the scion is effective in cleaving the viral genomic sequence. Thus, the present invention shows for the first time that a signal transferred from a rootstock to a scion interferes with a functional expression of a nucleic acid sequence that is not expressed by the plant genome, in a sequence-specific manner. This finding suggests that the transmitted signal is RNA. Smimov et al. (supra) showed that expression of pokeweed antiviral protein (PAP) in transgenic plants induces virus resistance in grafted plants. The authors further demonstrate that the resistance acquired by the scion is not dependent on salicylic acid accumulation and synthesis of pathogenesis-related proteins. However, these results differ substantially from the phenomena described by the present invention, as enzymatic activity of PAP is required for generating the signal that renders scion resistant to virus infection. In addition, the PAP activity confers non-specific viral resistance, as opposed to the sequence-specific resistance impart to the engrafted plants of the present invention.

According to one embodiment, the present invention discloses the protection of a susceptible tobacco scion (Nicotiana benthamiana) against the zucchini yellow mosaic virus (ZYMV) by grafting onto a transgenic resistant tobacco rootstock, wherein the resistant rootstock comprises a DNA construct designed for generating siRNAs targeted to a segment of the ZYMV genome, including the gene encoding for a coat protein and the 3′ non-translated region of the viral genome. This embodiment serves as a non-limiting example demonstrating the boarder scope of the invention as described herein above.

The compositions and methods of the present invention may be employed to confer resistance to any plant which is susceptible to viral infection. Non-limiting examples include plants of the Cucurbitaceae family, soybean, wheat, oats, sorghum, cotton, tomato, potato, tobacco, pepper, rice, corn, barley, Brassica, and Arabidopsis. According to one embodiment, the engrafted, virus-resistant plant is of the Cucurbitaceae family. According to one preferred embodiment, the transgenic virus-resistant plant is selected from the group consisting of watermelon, melon, pumpkin, squash, zucchini and cucumber. Virus specificity would be determined by the type and design of the nucleic acid sequence transformed into the plant. The nucleic acid sequence may encode a transcript targeted to silence one corresponding segment of the viral genome or more, and more than one nucleic acid sequence may be transformed into the plant.

According to certain embodiments, the present invention provides a plant comprising a transgenic rootstock comprising a nucleic acid sequence having at least 90% identity to at least one segment of a genome of a soil-borne virus as to be resistant to disease caused by the soil-borne virus and a scion susceptible to the disease, wherein the engrafted plant is protected from said disease caused by said soil-borne virus.

According to one embodiment, the plant is resistant to a disease caused by a soil-borne virus selected from the group consisting of, but not limited to, nematode-transmitted viruses: Nepoviruses: Arabis mosaic virus, Grapevine fanleaf virus, Tomato black ring virus, Raspberry ringspot virus, Tomato ringspot virus, and Tobacco ringspot virus; Tobraviruses: Pea early browning virus, Tobacco rattle virus and Pepper ringspot virus; fungal-transmitted viruses: Cucumber leafspot virus, Cucumber necrosis virus, Melon necrotic spot virus, Red clover necrotic mosaic virus, Squash necrosis virus, Tobacco necrosis satellite virus, Lettuce big-vein virus, Pepper yellow vein virus, Beet necrotic yellow vein virus, Beet soil-borne virus, Oat golden stripe virus, Peanut clump virus, Potato mop top virus, Rice stripe necrosis virus, Soil-borne wheat mosaic virus, Barley mild mosaic virus, Barley yellow mosaic virus, Oat mosaic virus, Rice necrosis mosaic virus, Wheat spindle streak mosaic virus and Wheat yellow mosaic virus; viruses transmitted via root wound: Tobamovirus genera: Tobacco mosaic virus, Tomato mosaic virus, Cucumber green mottle mosaic tobamovirus, Cucumber fruit mottle mosaic virus, Kyuri green mottle mosaic virus, Odontoglossum ringspot virus, Paprika mild mottle virus, Pepper mild mottle virus, Ribgrass mosaic virus and Tobacco mild green mosaic virus; and viruses transmitted by unknown rout: Watercress yellow spot virus, Broad been necrotic wilt virus, Peach rosette mosaic virus and Sugarcane chlorotic streak virus.

According to one embodiment, the transgenic rootstock comprises a nucleic acid sequence encoding a putative 54 kDa protein being a fragment of the replication protein of cucumber fruit mottle mosaic virus (CFMMV). According to one currently preferred embodiment, the transgenic rootstock comprises a nucleic acid sequence having the sequence set forth in SEQ ID NO:1.

According to additional embodiments, the present invention provides a plant comprising a transgenic rootstock comprising a DNA construct designed for generating siRNAs targeted to at least one segment of a viral genome as to be resistant to a disease caused by the virus and a scion susceptible to the disease caused by said virus, wherein the engrafted plant is resistant to said infection by said virus.

Plants can be designed to be resistant to any virus, depending on the targeted segment of the viral genome. According to certain embodiments, the plants are resistant to soil-borne viruses as well as to viruses transmitted by vectors which affect the aerial part of the plant, as described herein above.

According to one embodiment, the DNA construct designed for generating siRNAs targeted to at least one segment of a viral genome comprises:

(a) at least one plant expressible promoter operably linked to;

(b) a nucleic acid sequence encoding an RNA sequence that forms at least one double stranded RNA, wherein the double stranded RNA molecule comprises a first nucleotide sequence of at least 20 contiguous nucleotides having at least 90% sequence identity to the sense nucleotide sequence of the target segment of the viral genome and a second nucleotide sequence of at least 20 contiguous nucleotides having at least 90% sequence identity to the complementary sequence of the sense nucleotide sequence of said target segment of said viral genome; and optionally

(c) a transcription termination signal.

According to some embodiments, the DNA construct according to the present invention is designed to express a stem-loop RNA, comprising further to the first (sense) and the second (antisense) nucleotide sequences a spacer polynucleotide sequence, located between the DNA region encoding the first and the second nucleotide sequences. The length of the spacer polynucleotide sequence may vary according to the specific structure of the stem-loop RNA. Typically, the ratio of the spacer length to the first and second nucleotide sequences length is in the range of 1:5 to 1:10.

According to one preferred embodiment, the DNA construct designed for generating siRNAs targeted to at least one segment of a viral genome comprises:

(a) at least one plant expressible promoter operably linked to;

(b) a nucleic acid sequence encoding an RNA sequence that forms at least one double stranded RNA in the form of stem-loop, wherein the double stranded RNA molecule comprises a first nucleotide sequence of at least 20 contiguous nucleotides having at least 90% sequence identity to the sense nucleotide sequence of the target segment of the viral genome; a second nucleotide sequence of at least 20 contiguous nucleotides having at least 90% identity to the complementary sequence of the sense nucleotide sequence of said target segment of said viral genome; and a spacer sequence; and optionally,

(c) a transcription termination signal.

According to one embodiment, the spacer comprises a nucleotide sequence derived from a gene intron, known in the art to enhance the production of siRNAs. According to one embodiment, the spacer comprises a nucleotide sequence comprising an intron from the castor bean catalase gene, having the sequence set forth on SEQ ID NO:3.

The term “DNA construct” and “nucleic acid sequence” as used herein refers to a polynucleotide molecule comprising at least one polynucleotide that is expressed in a host cell or organism. Typically such expression is under the control of certain cis acting regulatory elements including constitutive, inducible or tissue-specific promoters, and enhancing elements. Common to the art, such polynucleotide sequence(s) are said to be “operably linked to” the regulatory elements. Nucleic acids sequences transferred into a eukaryotic cell typically also include eukaryotic or bacterial derived selectable markers that allow for selection of eukaryotic cells containing the nucleic acid sequence. These can include, but are not limited to, various genes conferring antibiotic resistance and various reporter genes, which are well known in the art. Optionally, the nucleic acid sequence further comprises cloning sites, one or more prokaryotic origins of replication, one or more translation start sites, one or more polyadenylation signals, and the like.

As used herein, the term “expression of a nucleic acid sequence”, or expression of a polynucleotide” refers to the process wherein a DNA region which is operably linked to appropriate regulatory regions, particularly to a promoter region, is transcribed into an RNA which is biologically active i.e., which is either capable of interaction with another nucleic acid or which is capable of being translated into a polypeptide or protein. It should be understood that according to the teaching of the present invention, translation of a protein is not necessarily required to obtain silencing of a viral gene or part thereof.

The term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and into protein, through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The nucleotide sequences of the present invention, being a segment of a viral genome, can be a full-length gene, a part thereof, a non-coding region or part thereof or a combination of same. As used herein, the term “homology” when used in relation to nucleic acid sequences refers to a degree of similarity or identity between at least two nucleotide sequences. There may be partial homology or complete homology (i.e., identity). “Sequence identity” refers to a measure of relatedness between two or more nucleotide sequences, expressed as a percentage with reference to the total comparison length. The identity calculation takes into account those nucleotide residues that are identical and in the same relative positions in their respective sequences. A gap, i.e. a position in an alignment where a residue is present in one sequence but not in the other is regards as a position with non-identical residues. Homology is determined for example using Gapped BLAST-based searches (Altschul et. al. 1997. Nucleic Acids Res. 25:3389-3402) and “BESTFIT”.

As used herein, “a complement of a nucleotide sequence” is the nucleotide sequence which would be capable of forming a double stranded DNA molecule with the nucleotide sequence, and which can be derived from the nucleotide sequence by replacing the nucleotide through their complementary nucleotide according to Chargaff's rules (AT; GC) and reading in the 5′ to 3′ direction, i.e. in opposite direction of the nucleotide sequence.

As used herein, nucleotide sequence of RNA molecule may be identified by reference to DNA nucleotide sequence of the sequence listing. However, the person skilled in the art will understand whether RNA or DNA is meant depending on the context. Furthermore, the nucleotide sequence is identical except that the T-base is replaced by uracil (U) in RNA molecule.

It will be appreciated that the longer the total length of the nucleic acid sequence homologous to the segment of the viral genome is, the requirements for sequence identity to the sequence of the segment of the viral genome are less stringent. The total nucleic acid sequence can have a sequence identity of at least about 90% with the corresponding segment of the viral genome, as well as higher sequence identity of about 95% or 100%.

According to certain embodiment, wherein the rootstock comprises a DNA construct designed for generating siRNAs targeted to at least one segment of the viral genome, the length of the second (antisense) nucleotide sequence of the DNA construct is largely determined by the length of the first (sense) nucleotide sequence, and may correspond to the length of the latter sequence. However, it is possible to use antisense sequences that differ in length by about 10%. Similarly, the nucleotide sequence of the antisense region is largely determined by the nucleotide sequence of the sense region, and may have a sequence identity of about 90% with the complement sequence of the sense region, as well as higher sequences identity of about 95% or 100%.

The first and the second nucleotide sequences of the DNA construct designed for generating siRNAs can be of any length providing the sequences comprising at least 20 contiguous nucleotides. Thus, the first and the second nucleotide can comprise a portion of a target sequence or a full length of the target sequence. According to some embodiments, the length of the nucleotides sequences is from 20 nucleotides to 1,200 nucleotides.

According to one embodiment, the siRNAs generated by the DNA construct of the present invention are targeted to silence a segment of the ZYMV genome, including the coding region for the viral coat protein.

According to one preferred embodiment, the first nucleotide sequence comprises a nucleotide sequence having 90% identity, preferably 95%, more preferably 100% identity to the nucleotide sequence set forth in SEQ ID NO:2 or a fragment thereof.

According to another preferred embodiment, the second nucleotide sequence comprises a nucleotide sequence having 90% identity, preferably 95%, more preferably 100% identity to the complement of the nucleotide sequence set forth in SEQ ID NO:2 or a fragment thereof.

According to certain embodiments, the first and the second nucleotide sequences are operably linked to the same promoter. The transcribed strands, which are at least partially complementary, are capable of forming dsRNA. According to other embodiments, the first and the second nucleotide sequences are transcribed as two separate strands. When the dsRNA is thus produced, the DNA sequence to be transcribed is flanked by two promoters, one controlling the transcription of the first nucleotide sequence, and the other that of the second, complementary nucleotide sequence. These two promoters may be identical or different. According to one embodiment, the first and the second nucleotide sequences are operably linked to the same promoter.

Plant expressible promoters are known in the art. The selection of a suitable promoter will be dictated by the plant species in which it is intended to use the DNA construct of the invention, availability, and required mode of action. Preferred promoters according to the teaching of the present invention are constitutive promoters, either general or tissue specific. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. Promoters often used for constitutive gene expression in plants include the CaMV 35S promoter, the enhanced CaMV 35S promoter, the Figwort Mosaic Virus (FMV) promoter, the mannopine synthase (mas) promoter, the nopaline synthase (nos) promoter, and the octopine synthase (ocs) promoter. A constitutive promoter isolated from strawberry vein banding virus (SVBV) isolated by inventors of the present invention and co-workers (Wang et al., 2000. Virus Genes 20:11-17) is preferably used.

The potential use of posttranscriptional gene regulation for suppression of specific genes has led to the development of various new methods for obtaining active siRNAs within a cell. For example, U.S. Application No. 20040262249 discloses the direct introduction of siRNA to silence a target gene, specifically a viral gene within a plant cell. The teaching of the present invention may be practiced with any method known in the art for the generation of siRNAs within a plant cell, or the direct introduction of siRNAs into the plant cell.

Nucleotide molecules which cross-hybridizes to the nucleic acid sequences described herein above, specifically to (i) a nucleic acid having a nucleotide sequence selected from SEQ ID NO:1 and SEQ ID NO:2 or (ii) the complement of a nucleotide sequence selected from SEQ ID NO:1 and SEQ ID NO:2 and fragments thereof are also within the scope of the present invention.

Successful hybridization is largely depends on the hybridization conditions. As used herein, the terms “stringent conditions” or “stringency”, refer to the conditions for hybridization as defined by the nucleic acid, salt, and temperature. These conditions are well known in the art and may be altered in order to identify or detect identical or related polynucleotide sequences. Numerous equivalent conditions comprising either low or high stringency depend on factors such as the length and nature of the sequence (DNA, RNA, base composition), nature of the target (DNA, RNA, base composition), milieu (in solution or immobilized on a solid substrate), concentration of salts and other components (e.g., formanide, dextran sulfate and/or polyethylene glycol), and temperature of the reactions (within a range from about 5° C. to about 25° C. below the melting temperature of the probe). One or more factors may be varied to generate conditions of either low or high stringency. Hybridization and wash conditions are well known and are exemplified in Sambrook et al., Molecular cloning: A laboratory manual, Second Edition, Cold Spring Harbor, N.Y. 1989, particularly chapter 11. According to one embodiment, cross-hybridization is performed under moderate stringency of 1.0-2.0×SSC at 65° C.

Constructs designed for transformation of nucleotide sequences into plant cells typically also include selectable markers that allow for selection of plant cells containing the construct of the invention. The term “selectable marker” refers to a gene which encodes an enzyme having an activity that confers resistance to an antibiotic or drug upon the cell in which the selectable marker is expressed, or which confers expression of a trait which can be detected (e.g., luminescence or fluorescence).

Typically, genes which confer antibiotic resistance, and which are well known in the art are used as a selectable marker. However, growth of genetically modified plants comprising gene(s) conferring antibiotic resistance in open fields, specifically of agricultural crops, is not allowed in a growing number of Western countries even when the final product does not contain the foreign genetic material. Thus, according to certain embodiments, a co-transformation method is utilized to select plants transformed with the DNA construct of the present invention.

According to one embodiment, co-transformation is performed with a DNA construct conferring viral gene silencing according to the present invention and a DNA construct comprising at least one selectable marker. Methods of co-transformation are known in the art, and are based in part on the finding that high percentage of the transformed cells bears both DNA constructs. Plants expressing the selectable marker are examined for the presence of the DNA construct conferring viral gene silencing, for example by PCR reaction. Selected plants are grown for maturity and are self-pollinated. In the resulted progeny, the two DNA constructs segregate independently, allowing selecting plants which comprise only the desired DNA construct.

Optionally, the nucleic acids sequence conferring viral gene cleavage comprises a transcription termination signal. A variety of terminators that may be employed in the constructs of the present invention are well known to those skilled in the art. The terminator may be from the same gene as the promoter sequence or from a different gene. According to one embodiment, the transcription termination signal is the NOS terminator.

According to additional aspect, the present invention provides a method for producing an engrafted plant resistant to infection by a virus comprising the steps of (a) providing a transgenic rootstock resistant to the viral infection other than by means of expression of an anti-viral protein; (b) providing a scion susceptible to infection by said virus; and (c) grafting the scion onto the rootstock as to obtain an engrafted plant resistant to said viral infection.

Grafting involves combining two independent plant parts into one plant. Such combination may be performed in various ways, including, but not limited to whip and tongue graft, splice graft, tip-cleft graft, side graft, saddle graft and bud graft (for further details see Garner R. J., The Grafter's Handbook, 5th Ed edition (March 1993) Cassell Academic; ISBN: 0304342742).

According to one embodiment, the rootstock is transformed with a nucleic acid sequence having at least 90% identity to at least one segment of a viral genome as to produce a transgenic rootstock resistant to the viral infection.

According to additional embodiment, the rootstock is transformed with a DNA construct designed for generating siRNAs targeted to at least one segment of a viral genome as to produce a transgenic rootstock resistant to the viral infection.

According to preferred embodiments of the invention, the affected segment of the viral genome is essential for the virus for plant infection and/or replication, so that its cleavage prevents the viral infection and/or replication, thereby providing a resistant plant.

Methods for transforming a rootstock with a nucleic acids sequence according to the present invention as to render the rootstock resistant to viral infection are known in the art. As used herein the term “transformation” or “transforming” describes a process by which a foreign DNA, such as a DNA construct, enters and changes a recipient cell into a transformed, genetically modified or transgenic cell. Transformation may be stable, wherein the nucleic acid sequence is integrated into the plant genome and as such represents a stable and inherited trait, or transient, wherein the nucleic acid sequence is expressed by the cell transformed but is not integrated into the genome, and as such represents a transient trait. According to preferred embodiments the nucleic acid sequence of the present invention is stably transformed into a plant cell.

There are various methods of introducing foreign genes into both monocotyledonous and dicotyledonous plants (Potrykus, I. 1991. Annu Rev Plant Physiol Plant Mol Biol 42, 205-225; Shimamoto, K. et al. 1989. Nature (1989) 338, 274-276).

The principal methods of the stable integration of exogenous DNA into plant genomic DNA include two main approaches:

Agrobacterium-mediated gene transfer: The Agrobacterium-mediated system includes the use of plasmid vectors that contain defined DNA segments which integrate into the plant genomic DNA. Methods of inoculation of the plant tissue vary depending upon the plant species and the Agrobacterium delivery system. A widely used approach is the leaf-disc procedure, which can be performed with any tissue explant that provides a good source for initiation of whole-plant differentiation (Horsch, R. B. et al. 1988. Plant Molecular Biology Manual A5, 1-9, Kluwer Academic Publishers, Dordrecht). A supplementary approach employs the Agrobacterium delivery system in combination with vacuum infiltration. The Agrobacterium system is especially useful for in the creation of transgenic dicotyledenous plants.

Direct DNA uptake. There are various methods of direct DNA transfer into plant cells. In electroporation, the protoplasts are briefly exposed to a strong electric field, opening up mini-pores to allow DNA to enter. In microinjection, the DNA is mechanically injected directly into the cells using micropipettes. In microparticle bombardment, the DNA is adsorbed on microprojectiles such as magnesium sulfate crystals or tungsten particles, and the microprojectiles are physically accelerated into cells or plant tissues.

Following stable transformation, plant propagation then occurs. The most common method of plant propagation is by seed. The disadvantage of regeneration by seed propagation, however, is the lack of uniformity in the crop due to heterozygosity, since seeds are produced by plants according to the genetic variances governed by Mendelian rules. In other words, each seed is genetically different and each will grow with its own specific traits. Therefore, it is preferred that the regeneration be effected such that the regenerated plant has identical traits and characteristics to those of the parent transgenic plant. The preferred method of regenerating a transformed plant is by micropropagation, which provides a rapid, consistent reproduction of the transformed plants.

Micropropagation is a process of growing second-generation plants from a single tissue sample excised from a selected parent plant or cultivar. This process permits the mass reproduction of plants having the preferred tissue and expressing a fusion protein. The newly generated plants are genetically identical to, and have all of the characteristics of, the original plant. Micropropagation allows for mass production of quality plant material in a short period of time and offers a rapid multiplication of selected cultivars with preservation of the characteristics of the original transgenic or transformed plant. The advantages of this method of plant cloning include the speed of plant multiplication and the quality and uniformity of the plants produced.

Micropropagation is a multi-stage procedure that requires alteration of culture medium or growth conditions between stages. The micropropagation process involves four basic stages: stage one, initial tissue culturing; stage two, tissue culture multiplication; stage three, differentiation and plant formation; and stage four, greenhouse culturing and hardening. During stage one, the tissue culture is established and certified contaminant-free. During stage two, the initial tissue culture is multiplied until a sufficient number of tissue samples are produced to meet production goals. During stage three, the newly grown tissue samples are divided and grown into individual plantlets. At stage four, the transformed plantlets are transferred to a greenhouse for hardening where the plants' tolerance to light is gradually increased so that they can continue to grow in the natural environment.

Those skilled in the art will appreciate that the various components of the nucleic acid sequences and the transformation vectors described in the present invention are operatively linked, so as to result in expression of said nucleic acid or nucleic acid fragment. Techniques for operatively linking the components of the constructs and vectors of the present invention are well known to those skilled in the art. Such techniques include the use of linkers, such as synthetic linkers, for example including one or more restriction enzyme sites.

As exemplified herein below, the transgenic rootstocks of the present invention express exogenous RNA molecules, specifically RNA sequences homologous to at least one segment of the genome of the target virus. The expression may be monitored by methods known to a person skilled in the art, for example by isolating RNA for the transgenic plant leave and testing for the presence of the viral RNA by employing specific primers in a polymerase chain reaction (PCR).

The present invention also relates to a plant cell or other parts thereof transformed with a nucleic acid sequence according to the present invention to serve as a rootstock. Furthermore, also encompassed by the present invention is a seed produced by the transgenic plant, wherein the seed also comprises the nucleic acid sequence transformed into the plant.

The transgenic resistant plant can be propagated for large-scale production of rootstocks by conventional breeding scheme. Furthermore, breeding can be used to introduce the DNA construct into other varieties of the same or related plant species, or in hybrid plants. Seeds obtained from the transgenic plants contain the nucleic acid sequence as a stable genomic insert, thus, also encompassed by the present invention are transgenic progeny of the transgenic plants described herein, grown from seeds of the transgenic plants. Any of the above-described transgenic plants can serve as a rootstock on which a scion is grafted according to the teaching of the present invention.

Selection of plants transformed with a nucleic acid sequence of the present invention as to provide transgenic resistant rootstock is performed employing standard methods of molecular genetic, known to a person of ordinary skill in the art. According to one embodiment, the nucleic acid sequence further comprises a nucleic acid sequence encoding a product conferring resistance to antibiotic, and thus transgenic plants are selected according to their resistance to the antibiotic. According to certain embodiments, the antibiotic serving as a selectable marker is one of the aminoglycoside group consisting of paromomycin and kanamycin. According to other embodiment, the nucleic acid sequence further comprises a reporter gene encoding a detectable product, and thus transgenic plants in which the product is detected are selected. According to certain embodiments, the reporter gene is selected from the group consisting of GUS, GFP and the like.

According to another embodiment, the plants transformed to provide resistant rootstocks are selected according to their resistance to viral infection. The virus selected to challenge the plants comprises in its genome the nucleic acid sequence transformed into the plants. According to one embodiment, the transgenic plants are selected according to their resistant to a soil-borne virus selected from the group consisting of, but not limited to, nematode-transmitted viruses: Nepoviruses: Arabis mosaic virus, Grapevine fanleaf virus, Tomato black ring virus, Raspberry ringspot virus, Tomato ringspot virus, and Tobacco ringspot virus; Tobraviruses: Pea early browning virus, Tobacco rattle virus and Pepper ringspot virus; fungal-transmitted viruses: Cucumber leafspot virus, Cucumber necrosis virus, Melon necrotic spot virus, Red clover necrotic mosaic virus, Squash necrosis virus, Tobacco necrosis satellite virus, Lettuce big-vein virus, Pepper yellow vein virus, Beet necrotic yellow vein virus, Beet soil-borne virus, Oat golden stripe virus, Peanut clump virus, Potato mop top virus, Rice stripe necrosis virus, Soil-borne wheat mosaic virus, Barley mild mosaic virus, Barley yellow mosaic virus, Oat mosaic virus, Rice necrosis mosaic virus, Wheat spindle streak mosaic virus and Wheat yellow mosaic virus; viruses transmitted via root wound: Tobamovirus genera: Tobacco mosaic virus, Tomato mosaic virus, Cucumber green mottle mosaic tobamovirus, Cucumber fruit mottle mosaic virus, Kyuri green mottle mosaic virus, Odontoglossum ringspot virus, Paprika mild mottle virus, Pepper mild mottle virus, Ribgrass mosaic virus and Tobacco mild green mosaic virus; and viruses transmitted by unknown rout: Watercress yellow spot virus, Broad been necrotic wilt virus, Peach rosette mosaic virus and Sugarcane chlorotic streak virus.

According to another embodiment, the transgenic plants are selected according to their resistant to a virus transmitted by a vector affecting the aerial part of the plant, selected from the group consisting of, but not limited to, a virus family: Caulimoviridae, Geminiviridae, Circoviridae, Reoviridae, Tartitiviridae, Bromoviridae, Comoviridae, Potyviridae, Tombusviridae, Sequiviridae, Clostroviridae and Luteoviridae; Tobamovirus, Tobravirus, Potexvirus, Carlavirus, Allexivirus, Capillovirus, Foveavirus, Trichovirus, Vitivirus, Furovirus, Pecluvirus, Pomovirus, Benyvirus, Hordeivirus, Sobemovirus, Marafivirus, Tymovirus, Idaeovirus, Ourmivirus, Umbravirus.

According to another aspect the present invention relates to the virus-resistant engrafted plants generated by the methods of the present invention. The plants, comprising a scion which is otherwise susceptible to a viral disease grafted onto a transgenic, resistant rootstock, are resistant to the viral disease. The rootstock comprises a nucleic acid sequence according to the invention stably integrated into its genome. The nature of the nucleic acid sequence, either a highly transcribed single transgene, or a transgene that produces siRNAs as described herein above, determines the resistance characteristics conferred to the engrafted plant. According to certain embodiments, the rootstock confers protection from disease caused by soil-borne viruses. According to other embodiments, the rootstock confers resistance to a disease caused by a virus transmitted by a vector affecting the aerial part of the plant.

The following non-limiting Examples herein below describe the resistant plants comprising a rootstock and scion according to the present invention. Unless stated otherwise in the Examples, all recombinant DNA and RNA techniques, as well as horticultural methods, are carried out according to standard protocols as known to a person with an ordinary skill in the art.

### EXAMPLES

### Experimental Procedures

### Construction of the binary vector pCAMSV 54-kDa

The putative 54-kDa encoding sequence with an additional 22 nucleotides upstream of the AUG at position 3629 (FIG. 1A), were cloned into the pCambia2301 binary vector (accession no. AF234316; Hajdukiewicz et al., 1994. Plant Mol Biol 25:989-994) using the full-length clone generated by Antignus et al., supra. The 54-kDa coding sequence was fused at its 5′-end to the non-coding region (NCR) of ZYMV and to the NOS poly-A terminator at its 3′-end (T) (FIG. 1B). The cloned gene and the NPTII marker gene were cloned downstream of the truncated Strawberry vein banding caulimovirus (SVBV) promoter (hence designated as SV) and the full-length SVBV promoter, respectively (Wang et al., 2000. Virus Genes 20:11-17 (FIG. 1B). This DNA construct was cloned between the T-DNA left (LB) and right (RB) borders (FIG. 1B). The SV promoter attached to the 5 NCR of ZYMV was PCR-amplified from the template ΔSVBVpr-ZYMV-FLC clone (Wang et al., supra) with the SV sense primer (SEQ ID NO:5: 5′CGCTAGCTATCACTGAAAAGACAGC3′) and the ZYMV NCR antisense primer harboring an NcoI site (underlined) (SEQ ID NO:6: 5′ GGCCATGGTTATGTC TGAAGTAAACG 3′). The PCR fragment (470 bp) was cloned into the pGEM-T vector (Promega, Madison, Wis., USA) and further designated pΔSVBV-NCRzy.

The 54-kDa coding sequence was amplified by PCR from clone pUC3′-3.3 kb with the following primers: 5′CGGCCATGGCATCGAAGGCGGGTTTTTGGACG3′ (54-kDa sense with NcoI site underlined, SEQ ID NO:7), 5′GAGGTGACCTAGACACTAGGCTTAATGAATAG3′ (54-kDa antisense with BstEII site underlined, SEQ ID NO:8). The putative 54-kDa PCR fragment (1461 bp) was previously digested by NcoI/BstEII and cloned into pΔSVBV-NCRzy digested by NcoI/BstEII. The obtained clone pΔSVBV-NCR54-kDa was double digested by EcoRl/BstEII and the resulting insert was cloned into the binary vector pCAMBIA2301, which was double digested with EcoRl/BstEII prior to cloning. The 35S promoter (located upstream of the NPTII gene) from pCAM35S-SV54-kDa was replaced by the intact SVBV promoter as follows: the SVBV promoter, previously cloned into pGEM-T and termed SVBVpr (Want et al., supra), was excised by double digestion of EcoRl/BglII sites, and cloned into the same site in the binary vector, now designated pCAMSV54-kDa. This final construct (FIG. 1B) was introduced into Agrobacterium tumefaciens EHA105 strain and used to transform the desired plant.

### Construction of the DNA Construct for Generating siRNAs, pCddCP-ZY

The 3′end of the ZYMV genome, including the intact gene encoding the virus coat protein and the 3′ non-coding region (NCR) (SEQ ID NO:2, accession No. M35095) was PCR amplified with primers harboring a BamHI and KpnI sites (sense primer 5′ATGGATCCCTGCAGTCAGGCACTCAGCCAACTGTGGC3′ SEQ ID NO:10) and the anti-sense primer harbored a NarI and PstI sites (5′ATGGCGCCGGTACCAGGCTTGCAAACGGAGTC3′ SEQ ID NO:11). This segment was isolated from a ZYMV Israel isolate, and is homologous to the nucleic acids sequence from position 8538 to 9588 of the ZYMV genome having accession number NC_{—}0033224 (SEQ ID NO:9).

Previously, a catalase intron was cloned into the polylinker of KS Bluescript. The cloned catalase intron contained BamnHI and PstI sites at the 5′end, and NarI and KpnI at the 3′ end. The PCR product of the 3′ end of the ZYMV genome (1050 bp) was first cut with KpnI and NarI, and the resulting product cloned downstream (3′ end) of the catalase intron in KS plasmid. Subsequently, the PCR product was cut with BamHI and PstI, and the product was cloned into the 5′end of the catalase intron in the KS plasmid. This resulted in an inverted repeat of the 3′end of the ZYMV genome in KS, separated by the catalase intron. The new clone was designated pKSddCP-ZY. The 35S CaMV promoter from a pCambia binary vector 2301 was replaced by the SVBV promoter. The construct described previously, pKSddCP-ZY, was cut by BamHI and KpnI, and cloned into the appropriate sites BamHI and KpnI downstream from the SVBV promoter, and upstream from a NOS terminator. The new clone was designated pCddCP-ZY (FIG. 2).

### Agrobacterium-Mediated Transformation

Agrobacterium cultures were grown overnight at 28° C. in LB medium containing appropriate selective antibiotics and 100 μM acetosyringone, and then sub-cultured for 4 h under the same conditions in medium without antibiotics. Bacteria were sedimented and re-suspended in liquid MS medium (Murashige, T. and Skoog, F. 1962. Physiology Plantarum 15:473-497) containing 3% sucrose at a final density of 0.5 OD. The transformation method was as previously described (Tabei et al., 1998. Plant cell report 17: 159-164), with several modifications.

### Transformation of Cucumber

Peeled cucumber seeds of cv. ‘Ilan’ (Zeraim Gedera Co., Israel) were surface sterilized by incubation for 1 min in 70% ethanol and then in 2% hypochlorite solution for 20 min. Following extensive washing, the seeds were incubated for 1-2 days on MS medium containing 3% sucrose and 0.8% Oxoid agar, at 25° C. in the dark. Seed embryos were dissected out and individual cotyledons were incubated for 1-2 days at 25° C. in the dark, on regeneration medium (MS medium, 3% sucrose, 2 mg/l benzylaminopurine (BAP), 1 mg/l abscisic acid (ABA), 0.8% Oxoid agar) supplemented with 200 μM acetosyringone. The cotyledons were dipped in Agrobacterium suspension for 5 min, dried on filter paper and returned to the same plates for 2 days of co-cultivation in the dark. Explants were then transferred to selection medium (regeneration medium supplemented with 500 mg/l Cefatoxim and 100 mg/l kanamycin) and incubated in a 16/8-h photoperiod regime with biweekly subcultures. Regenerated shoots were excised and transferred to elongation medium (MS, 3% sucrose, 1 mg/l gibberelic acid, 0.1 mg/l BAP, 0.1 mg/l ABA, 0.8% Oxoid agar, 500 mg/l Cefatoxim and 100 mg/l kanamycin). Rooting of shoots was induced in MS, 3% sucrose, 0.5 mg/l indole butyric acid, 0.8% Oxoid agar, 500 mg/l Cefatoxim and 100 mg/l kanamycin. Rooted plantlets were transplanted in Jiffy 7 peat pellets for hardening, before being transferred to a greenhouse for further growth.

### Transformation of Tobacco

Leaf explants from axenic plants were incubated for 1-2 days at 25° C. in the dark, on regeneration medium (MS medium, 3% sucrose, 1 mg/1 benzylaminopurine (BAP), 0.1 mg/l naphthalene acetic acid (NAA), 0.8% Oxoid agar) supplemented with 200 μM acetosyringone. The explants were dipped in Agrobacterium suspension for 5 min, dried on filter paper and returned to the same plates for 2 days of co-cultivation in the dark. Explants were then transferred to selection medium (regeneration medium supplemented with 500 mg/l Cefatoxim and 250 mg/l kanamycin) and incubated in a 16/8-h photoperiod regime with biweekly subcultures. Regenerated shoots were excised and transferred to rooting medium (MS, 3% sucrose, 0.8% Oxoid agar, 500 mg/l Cefatoxim and 250 mg/l kanamycin). Rooted plantlets were transplanted in Jiffy 7 peat pellets for hardening, before being transferred to a greenhouse for further growth.

### Segregation Assay in R₁ Seedlings

Progenies from individual transformed plants were screened for segregation of the DNA construct, as follows: R₁ seeds were surface sterilized and germinated as described above, in the presence of 100 mg/l kanamycin. Transgenic seeds (harboring the NPT II gene) showed normal root development, whereas non-transgenic offspring plants were easily detected according to their atrophied and unbranched roots. The kanamycin-resistant/-susceptible ratio was recorded, and transgenic seedlings were utilized in further experiments.

### Evaluation of the Resistance Response

Kanamycin-resistant R₁ seedlings were screened under greenhouse conditions for virus resistance.

Resistance to CFMMV

Kanamycin-resistant cucumber plants were inoculated mechanically with purified CFMMV at 1 mg/ml in 50 mM phosphate buffer (pH 7.4), or with viral RNA at 400 μg/ml in 50 mM phosphate buffer (pH 8.0). For most transgenic progenies, more than 10 seedlings were initially screened by inoculation. Inoculated seedlings were kept for several weeks under greenhouse conditions. Responses to inoculation were determined by visual inspection of symptoms, and for the presence of CFMMV by DAS-ELISA with a specific antiserum prepared at 1:1,000 dilution (Antignus et al., supra). Further resistance analysis was performed by mechanical back inoculation to N. benthamiana and Datura stramonium three weeks post inoculation.

Resistance to ZYMV

Kanamycin-resistant GUS-expressing tobacco plants were inoculated mechanically with sap diluted at a 1:5 ratio, from tobacco plants co-infected with ZYMV and a tobamovirus tentatively identified as cucumber green mottle mosaic virus (CGMMV), that served as helper to obtain systemic spread of ZYMV. For most transgenic progenies, more than 10 seedlings were initially screened by inoculation. Inoculated seedlings were kept for several weeks under greenhouse conditions. Responses to inoculation were determined by ELISA with a specific ZYMV-CP antiserum prepared at 1:2,000 dilution.

Cucumber seedlings at the cotyledon stage were used for grafting. A top grafting method was employed, in which the non-transformed scion was grafted following a diagonal cut of the stem under the cotyledonary node and installed on top of a transgenic or non-transformed rootstock seedling, which was also cut diagonally at the cotyledonary node in a manner that retained a single cotyledon (so that the grafted seedling contained three cotyledons). Three weeks old tobacco seedlings were top grafted by making a diagonal cut at the rootstock stem and installing a scion which was similarly diagonally cut. The scion/rootstock junction was secured in place with small plastic clips. During the first week the grafted plants were maintained in high humidity.

### Inoculation of Plants

Cucumber seedlings were used as source plants to maintain cultures of CFMMV KGMMV, ZGMMV CGMMV and cucumber vein yellowing virus (CVYV). Squash plants were used as the source of inoculum for ZYMV and CMV-Fny. Inocula were prepared by grinding young leaves of source plants in distilled water.

Cotyledons (3 days post emergence) were mechanically inoculated following dusting with carborundum. Root inoculation was carried out by transplanting the tested plants from polystyrene trays into plastic pots (10 cm diameter) containing virus-infested perlite medium. Crude inoculum was prepared by grinding virus-infected cucumber leaves in 0.01 M phosphate buffer pH 7 at a ratio of 1:100. Graft inoculation was conducted by making a diagonal cut in the stem of a virus-infected plant at the stage of four true leaves. The apical part of the plant to be inoculated was removed and the lower part of its stem was trimmed to a wedge shape before being inserted into the diagonal cut of the infected rootstock. The contact point of the scion and rootstock was bound with a parafilm strip to provide good fusion. During the first week grafted plants were maintained in plastic bags to maintain high humidity.

Virions were partially purified from infected plants as described (Chapman S. N. 1998. in: Plant Virology Protocols. G. D. Foster and S. C. Taylor, Eds. Humana Press, New Jersey: 123-129). Partially purified virions were mixed 1:1 with 2×SDS-PAGE loading buffer and boiled for 5 min. (Sambrook et al., 1989. Molecular cloning-A LABORATORY MANUAL, Second Edition). The boiled material (10 μl) was fractionated by SDS-PAGE on a 12.5% polyacrylamide gel.

### Extraction and Analysis of RNA, Northern Blotting and RT-PCR

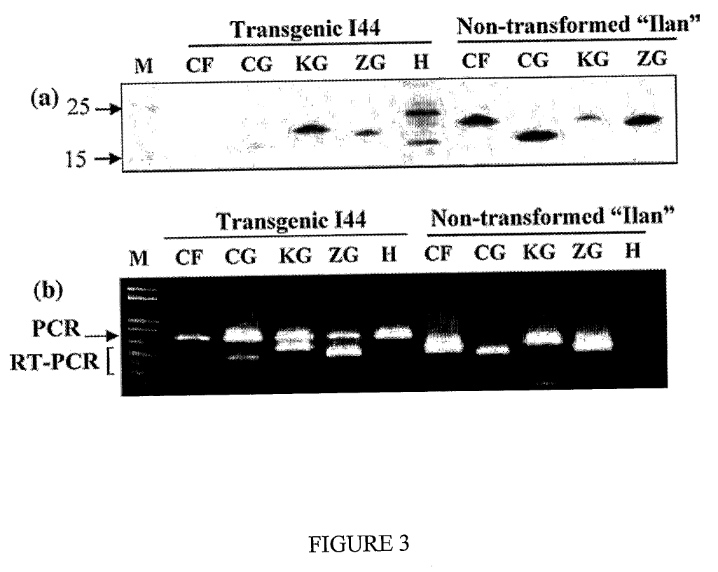

Transcription of the viral RNA was determined by Northern blotting and RT-PCR analysis of total RNA extracted from seedlings three weeks after germination. Young leaf tissue (300 mg) was ground to a fine powder in liquid nitrogen and RNA was extracted with the TRI-REAGENT kit (Molecular Research Center, Inc., Cincinnati, Ohio, USA), according to the manufacturer's instructions. RNA concentration was measured by GeneQuant (Pharmacia Biotech), and comparable amounts of RNA from different sources were loaded on a gel. About 30 μg of each sample were run in a denaturing 1.5% agarose gel containing formaldehyde. The RNA separated in the gel was then blotted onto Hybond-NX membranes (Amersham, NJ, USA) and fixed by exposure to a 80 W UV lamp (Vilber Lourmat BLX-254, France) for 2 min. Prehybridization with Rapid-hyb buffer (Amersham Pharmacia) was performed for 2 h. CFMMV RNA was detected in transformed plants by hybridization with ³²P-labeled cDNA probe of the 54-kDa gene of CFMMV (nucleotides 3824-4693) with a randomly primed ³²P-labeled DNA probe (Random primer DNA labeling mix kit; Biological Industries, Beit HaEmek, Israel). RT-PCR for detection of CFMMV RNA in inoculated plants was conducted in a one-tube single-step method with 2-5 μg total RNA according to Arazi et al. (2001. J Biotechnol 87:67-82) with specific primers of the 54-kDa gene (sense: 5′GCTACGGAGCGTCCGCGG3′, SEQ ID NO:12, and antisense: 5′CGCGGTCGACTGTATGTCAT3′, SEQ ID NO:13). RT-PCR cycles were as follows: 46° C. 30 min; 94° C. 2 min, followed by 35 cycles at 94° C., 58° C. and 72° C., each of 30 seconds, and one final cycle of 5 min at 72° C. RT-PCR for infectivity assay of various tobamoviruses (FIG. 3) were performed in two steps with specific primers of the CP genome of the following viruses:

CGMMV (5′TCTGACCAGACTACCGAAAA3′, SEQ ID NO:14 and 5′ATGGCTTACAATCCGATCAC3′, SEQ ID NO:15);

KGMMV (5′GAGAGGATCCATGTTTCTAAGTCAGGTCCT3′, SEQ ID NO:16 and 5′GAGAGAATTCTCACTTTGAGGAAGTAGCGCT3′, SEQ ID NO: 17);

ZGMMV (5′TCTATCGCTTAACGCAGC3′, SEQ ID NO: 18 and 5′ATGTCTTACTCTACTTCTGG3′, SEQ ID NO:19); and

CFMMV (5′CAAGACGAGGTAGACGAAC3′, SEQ ID NO:20 and 5′ATGCCTTACTCTACCAGCG3′, SEQ ID NO:21). RT-PCR was performed by RT-PCR AmpTaq kit (Perkin Elmer) in an i-cycler (Bio-Rad) and cycling step was 37° C. for 1 hr and 30 cycles of 1 min at 94° C., 40 s at an annealing temperature of 53° C. (KGMMV), 52° C. (ZGMMV, CGMMV) or 44° C. (CFMMV), 1 min at 72° C., and finally 72° C. for 10 min.

### DNA Extraction and PCR Analysis

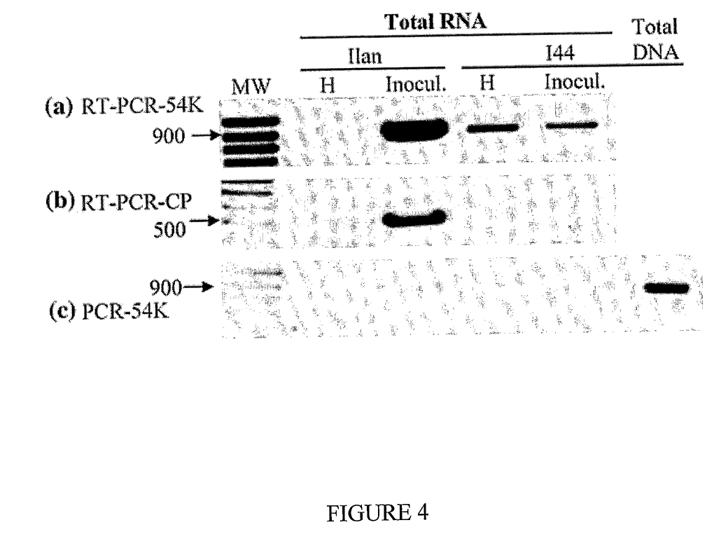

Total genomic DNA was extracted from young leaves (3 weeks after germination) by the CTAB method (Chen, D. H. and Ronald, P. C. 1999. Plant Mol Biol Reporter 17:53-57). DNA solution (1 μl) was diluted in 25 μl of a PCR reaction mixture, containing primers according to the sequence of CFMMV 54-kDa gene (accession no. AF321057, SEQ ID NO:4). Two sets of primers were used for detection of the 54-kDa gene: the first primer set at positions 3824 and 4693 (SEQ ID NO:12 and SEQ ID NO:13; FIG. 4) and a second primer set at positions 3785 and 4479 (5′GAAAAAGGAGTTTTTGATCCCGCT3′, SEQ ID NO:22 and 5′ACTGATATGCGTCTTCTTATGCCC3′, SEQ ID NO:23; FIG. 3). PCR conditions were: one cycle of 2 min at 94° C. and 35 cycles at 94, 58 and 72° C., each of 30 sec, and finally 5 min at 72° C.

### Example 1

### Identification and Characterization of Resistant Cucumber Lines

Following Agrobacterium-mediated transformation with the pCAMSV 54-kDa construct, individual R₀ transformants were grown to maturity and selfed to obtain R₁ seeds. The presence of CFMMV 54-kDa gene within the plant genome was confirmed by PCR analysis for all of the kanamycin-resistant R₁ lines indicated in Table 1. Following a resistance screening, eight out of 14 R₁ lines exhibited full resistance response (Table 1). Some of the remaining lines were fully susceptible; others were characterized as partially resistant. No attempt was made to evaluate accumulation of CFMMV RNA in the inoculated cotyledons. However, no virus accumulation was detected in upper leaves of the eight resistant replicase lines, as shown in most lines by ELISA and back-inoculation assays. Similarly, the resistance response remained unaltered when plants were grown at higher at higher temperatures (30-35° C.).

TABLE 1
Screening for resistance to CFMMV in R1 transgenic cucumber
lines containing the 54-kDa gene

| | | | Response to back |
|---|---|---|---|
| Line^{a} | Infection rate^{b} | ELISA | inoculation^{c} |
| R44 | 0/64 | − | − |
| R45 | 0/7 | − | − |
| R84 | 0/5 | − | n.t |
| R169 | 0/9 | − | − |
| R175 | 0/17 | − | − |
| R179 | 0/10 | − | − |
| R187 | 0/3 | − | n.t |
| R205 | 0/8 | − | − |
| R28 | 9/9 | + | n.t |
| R146 | 6/6 | + | n.t |
| R149 | 2/5 | + | n.t |
| R170 | 9/9 | + | n.t |
| R181 | 7/10 | + | n.t |
| R189 | 9/12 | + | n.t |
| ‘Ilan’ (control) | 50/50 | + | n.t |

^{a}Each numbered line represents the progeny of individual R₀ transgenic plants. ‘Ilan’ is the parental non-transformed cultivar.
^{b}Kanamycin-resistant R₁ seedlings were evaluated for resistance to CFMMV by mechanical inoculation with purified virus at 1 mg/ml. The number of susceptible seedlings out of the total inoculated is shown (infected/inoculated). Fully resistant lines are indicated in boldface.
^{c}Seedlings showing no symptoms were assayed for CFMMV accumulation by back inoculation to N. benthamiana. Systemic symptoms (+) or lack of symptoms (−) on N. benthamiana were recorded three weeks post inoculation. n.t.—Not tested.

### Example 2

### Characterization of the Resistance in the Homozygous 144 Line

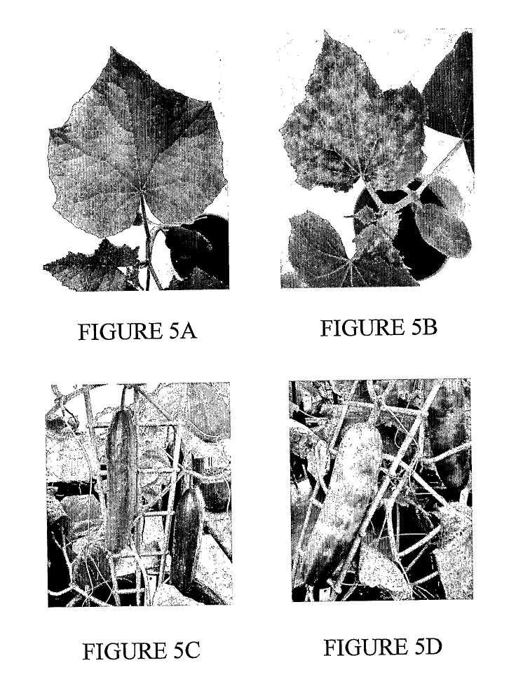

Further extensive studies were performed to characterize the resistance of line R44 (Table 1), which shows the putative expected segregation by a single NPT II insert. For genetic consistency, seeds from ten different R₁ plants of line R44 were germinated in the presence of kanamycin as described herein above and a R₂ homozygotic line (hence designated as line I44) that does not segregate for the transgenic locus was identified and used for further studies. 144 plants exhibited a normal phenotype and normal fruit development, indistinguishable from the original cultivar (‘Ilan’). The parental line (‘Ilan’) was highly susceptible to CFMMV inoculation (Table 2) and developed strong mosaic symptoms 14 days after inoculation, with subsequent leaf deformations, plant stunting, and abnormal fruits with yellow patches (FIG. 5). In contrast, I44 plants were fully resistant to mechanical inoculation with plant extract (Table 2, FIG. 5) and purified RNA (data not shown). Soil-mediated inoculation of CFMMV in the greenhouse is a triggering factor for the onset of viral epidemics (Antignus, unpublished). Therefore, the response of 144 seedlings planted in soil deliberately infested with CFMMV inoculum was examined. Moreover, line I44 was also challenged by a most aggressive inoculation method, namely, grafting on top of an infected susceptible rootstock (cv. ‘Ilan’). 144 plants remained symptomless, and no virus accumulation could be detected either by ELISA or by back-inoculation to susceptible hosts (N. benthamiana and cucumber), irrespective of the inoculation method used (Table 2).

TABLE 2
Response of line I44 to mechanical, soil and graft inoculation with
CFMMV.

| Inoculation | | Infection | | Back |
|---|---|---|---|---|
| Method | Genotype | Rate^{b} | ELISA | Inoculation^{c} |
| Mechanical | I44 | 0/65 | − | − |
| | Ilan | 30/30 | + | + |
| | I44 | 0/10 | − | − |
| Soil | Ilan | 8/10 | + | n.t |
| Grafting^{a} | I44 (scion) | 0/6 | − | − |
| | Ilan (scion) | 6/6 | + | n.t |

^{a}Graft inoculation was effected by grafting I44 or ‘Ilan’ scions on top of infected ‘Ilan’ rootstock.
^{b}Infectivity rates in I44 and non-transformed ‘Ilan’ plants were scored 4 weeks post inoculation as number of symptomatic plants out of the total number of inoculated plants.
^{c}Back inoculation performed by mechanical inoculation of N. benthamiana with sap extracted from inoculated plants 4 weeks post inoculation. n.t = not tested

### Example 3

### Molecular Characterization of the I44 Resistant Line

The presence of the viral nucleic acids sequence in the genome of line I44 was verified by PCR (FIG. 4C) and Southern blotting (data not shown). Transcription of the 54-kDa coding sequence was detected by RT-PCR in inoculated or non-inoculated I44 plants (FIG. 4A). On the contrary, the PCR reaction with the total RNA preparation from I44 plants was negative (FIG. 4C), indicating that the amplified RT-PCR band shown in FIG. 4A was not due to contaminating DNA residues in the RNA preparation. An RT-PCR assay was performed to assess whether the lack of symptoms in line I44 inoculated with CFMMV was due to lack of virus accumulation. No amplified band was observed with specific primers for CFMMV coat protein (CP) in the I44 line, in contrast to the presence of a positive CP band in ‘Ilan’ inoculated plants (FIG. 4B). These results further confirm the observation that no trace of virus accumulation can be detected in I44 plants.

### Example 4

### Screening for Resistance Against Other Tobamoviruses

It has been previously shown that replicase-mediated resistance exhibits high sequence specificity. Therefore, the response of the I44 line against infection with various cucurbit-infecting tobamoviruses was examined. Line I44 and the non-transformed ‘Ilan’ cultivar were inoculated with three additional tobamoviruses: KGMMV, ZGMMV and CGMMV. The non-transformed cv. ‘Ilan’ showed symptoms with KGMMV and ZGMMV starting at 8 days post inoculation (dpi), and 2 days later with CFMMV and CGMMV (Table 3). A significant delay of symptom appearance was observed in line I44: symptoms were visible only at 14 dpi with CGMMV and ZGMMV, and at 20 dpi with KGMMV. In addition, a marked attenuation of symptoms was observed in I44 plants infected with CGMMV throughout the entire experiment (30 dpi), in contrast to the severe symptoms exhibited by non-transformed plants (Table 3). The response of line I44 to tobamovirus infections was confirmed by SDS-PAGE for detection of purified virions, and by RT-PCR for viral RNA (FIG. 3). The accumulation of ZGMMV, CGMMV and KGMMV virions was clearly detected both in I44 and control ‘Ilan’ plants; however, CFMMV virions were detected only in these later plants (FIG. 3A). In addition, while viral RNA of CGMMV, KGMMV and ZGMMV was detected by RT-PCR in I44 plants, as well as in the non-transformed control plants, CFMMV RNA was only found in the control ‘Ilan’ plants (FIG. 3B).

TABLE 3
Response of I44 plants to infection with various cucurbit
tobamoviruses

| | Transgenic line-I44 | | | | Non-transformed “Ilan” | | | |
|---|---|---|---|---|---|---|---|---|
| dpi^{a} | CFMMV | CGMMV | KGMMV | ZGMMV | CFMMV | CGMMV | KGMMV | ZGMMV |
| 8 | − | − | − | − | − | − | ++++ | ++++ |
| 10 | − | − | − | − | +++ | +++ | +++++ | +++++ |
| 14 | − | + | − | ++ | ++++ | ++++ | +++++ | +++++ |
| 18 | − | + | − | ++++ | +++++ | +++++ | +++++ | +++++ |
| 20 | − | + | ++ | ++++ | +++++ | +++++ | +++++ | +++++ |
| 24 | − | + | ++++ | +++++ | +++++ | +++++ | +++++ | +++++ |
| 26 | − | + | +++++ | +++++ | +++++ | +++++ | +++++ | +++++ |
| 30 | − | + | +++++ | +++++ | +++++ | +++++ | +++++ | +++++ |

^{a}Cucumber plants were inoculated at the cotyledon stage, and the severity of symptoms (mosaic spread and stunting) was scored from 1+ to 5+ for a period of 30 days post inoculation (dpi). (−) No symptoms. The data are summarized from two independent experiments with 10 plants for each treatment.

### Example 5

### Transcription of the Transformed Nucleic Acid Sequence in Virus-Inoculated Plants

The specificity and the remarkable resistance exhibited by line I44 to CFMMV infection might indicate the prevalence of an RNA-mediated resistance mechanism, possibly associated with RNA silencing. To test this hypothesis, total RNA was extracted from line I44 and from ‘Ilan’ plants, before and after inoculation with CFMMV. Samples with an equivalent amount of total RNA were analyzed by Northern blot hybridization with a labeled 54-kDa probe. The probe hybridized with CFMMV genomic RNA (upper band), the putative subgenomic RNA I1 harboring the 54-kDa, and with the 54-kDa transgene transcripts. As expected, the transcript in I44 plants was shorter than the I1 subgenomic RNA detected in the infected control plants (FIG. 6). The 54-kDa transcripts were observed only in line I44 plants, and the level of transcript accumulation was not substantially affected by prior inoculation of I44 plants with CFMMV or ZYMV (FIG. 6). It has been shown that silencing-mediated viral resistance can be suppressed by inoculation with potyviruses prior to challenging the plants with the virus (Savenkov, E. I. and Valkonen, J. P. 2002. J Gen Virol 83:2325-2335), or by changing temperature conditions Szittya et al., 2003. EMBO J 22:633-640). Plants of line I44 were evaluated for resistance to CFMMV, following pre-inoculation with one of the following potyviruses: ZYMV, Zucchini fleck mosaic virus (ZFMV) and Cucumber vein yellowing virus (CVYV, genus Ipomovirus; family Potyviridae), or with Cucumber mosaic virus (CMV) (Table 4). I44 plants showed typical symptoms of individual potyviruses or CMV, but remained resistant to CFMMV in the sequential infections, as confirmed by ELISA and back-inoculation to Datura stramonium. In addition, the resistance of line I44 to CFMMV infection was not affected by growing the inoculated plants in growth chambers at different temperatures (20, 28 or 35° C.).

TABLE 4
I44 resistance to CFMMV infection following inoculation with other
viruses

| | ZYMV + | ZYFV + | CVYV + | CMV + | CFMMV + |
|---|---|---|---|---|---|
| Line | CFMMV | CFMMV | CFMMV | CFMMV | CFMMV |
| I44 | 0/6 | 0/6 | 0/6 | 0/6 | 0/6 |
| ‘Ilan’ | 4/4 | 6/6 | 6/6 | 4/4 | 6/6 |

### Example 6

### Protection of Susceptible Scions by a I44 Rootstock

Tobamovirus particles survive for long periods in the soil, and CFMMV infection through the roots in infested soils is a common phenomenon. Since line I44 showed resistance to soil inoculation with CFMMV (Table 2), we tested the suitability of this line to serve as a protective rootstock for non-transformed scions.

Control Ilan plants were grafted onto line I44 or Ilan rootstocks and planted in soil infested with CFMMV. Most of the plants (12/16) grafted on non-transformed Ilan rootstocks showed clear symptoms of CFMMV, and tested positive by ELISA. However, none of the scions (0/16) grafted on I44 became infected throughout the duration of the experiment (5 weeks), as assessed by visual symptoms, ELISA tests and back-inoculation to N. benthamiana. In parallel experiments, ‘Ilan’ scions grafted onto I44 rootstock were susceptible to direct mechanical inoculation with CFMMV.

### Example 7

### Protection of Susceptible Scions by Resistant N. benthamiana Rootstock

N. benthamiana leaf disks were transformed with Agrobacterium tumefaciens harboring the pCddCP-ZY construct, as described herein above. Following selection on regeneration media, individual putative transformants were identified by GUS expression. Confirmed transformants were selfed to produce R₁ generation. Progenies from individual transformed plants were screened for segregation of the DNA construct, as follows: R₁ seeds were surface sterilized and germinated in the presence of 250 mg/l kanamycin. Transgenic seeds (harboring the NPT II gene) showed normal root development, whereas non-transgenic offspring plants were easily detected according to their atrophied and unbranched roots. The kanamycin-resistant/susceptible ratio was recorded, and transgenic seedlings were utilized in inoculation experiments.

Kanamycin-resistant GUS-expressing tobacco R1 seedlings were inoculated mechanically with sap diluted at a 1:5 ratio, from tobacco plants co-infected with ZYMV and a tobamovirus tentatively identified as cucumber green mottle mosaic virus (CGMMV), that served as helper to obtain systemic spread of ZYMV. For most transgenic progenies, more than 10 seedlings were initially screened by inoculation. Inoculated seedlings were kept for several weeks under greenhouse conditions. Response to inoculation was determined by ELISA with a specific ZYMV-Coat Protein antiserum prepared at a dilution of 1:2,000.

Selected transgenic lines were used as rootstock in subsequent grafting experiments. Three-week-old tobacco seedlings were top grafted by making a diagonal cut at the rootstock stem and installing a scion that was similarly diagonally cut. The scion/rootstock junction was secured in place with small plastic clips. During the first week the grafted plants were maintained in high humidity. Mechanical inoculation of the scions were performed 3 to 4 weeks post-grafting with sap diluted at a 1:5 ratio, from tobacco plants co-infected with ZYMV and a tobamovirus as described herein above. Inoculated grafted plants were kept for several weeks under greenhouse conditions. Response to inoculation was determined by ELISA with a specific ZYMV-CP antiserum prepared at a dilution of 1:2,000. In some experiments, the presence of ZYMV in inoculated scions was determined by back inoculation to susceptible squash plants. The results are summarized in table 5 below.

TABLE 5
Resistance to ZYMV imparted to a scion by transgenic rootstock

| | Number of Infected |
|---|---|
| Rootstock | scions/total plant number |
| Transgenic lines | |
| Z89 | 0/12 |
| Z99 | 0/4 |
| Z102 | 0/9 |
| Z97 | 1/8 |
| Z100 | 1/6 |
| Z101 | 1/12 |
| Total | 3/51 (6%) |
| Non-transgenic controls | |
| Grafted | 7/10 (70%) |
| Non-grafted | 17/19 (89%) |

Non-grafted tobacco controls or scions grafted on non-transgenic rootstocks exhibited a high infection rate of 89% and 70%, respectively. The response of scions grafted on transgenic lines may be divided into 2 groups: those grafted onto rootstock lines Z89, Z99 or Z102 were fully protected against ZYMV systemic infection; from those grafted onto rootstock lines Z97, Z100 or Z101 few exhibited the disease symptoms. In few protected rootstocks, the absence of ZYMV in inoculated scions was confirmed by back inoculation to squash plants.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1-67. (canceled)

68. A plant comprising a transgenic rootstock resistant to a viral disease other than by means of expression of an anti-viral protein and a scion susceptible to the viral disease, wherein the engrafted plant is resistant to said viral disease.

69. The plant according to claim 68, wherein the transgenic rootstock resistant to a viral disease comprises a nucleic acid sequence having at least 90% identity to at least one segment of the viral genome.

70. The plant according to claim 69, wherein the transgenic rootstock resistant to a viral disease comprises a DNA construct designed for generating siRNAs targeted to the at least one segment of the viral genome.

71. The plant according to claim 69, wherein the at least one segment of the viral genome encodes a viral protein or parts thereof, selected from the group consisting of a viral coat protein, a viral replication protein, a viral movement protein or parts thereof.

72. The plant according to claim 71, wherein the viral protein is a viral replication protein or part thereof.

73. The plant according to claim 72, wherein the transgenic rootstock is resistant to a disease caused by a soil-borne virus, and wherein said plant is protected from a disease caused by a soil-borne virus selected from the group consisting of nematode-transmitted viruses: Nepoviruses: Arabis mosaic virus, Grapevine fanleaf virus, Tomato black ring virus, Raspberry ringspot virus, Tomato ringspot virus, and Tobacco ringspot virus; Tobraviruses: Pea early browning virus, Tobacco rattle virus and Pepper ringspot virus; fungal-transmitted viruses: Cucumber leafspot virus, Cucumber necrosis virus, Melon necrotic spot virus, Red clover necrotic mosaic virus, Squash necrosis virus, Tobacco necrosis satellite virus, Lettuce big-vein virus, Pepper yellow vein virus, Beet necrotic yellow vein virus, Beet soil-borne virus, Oat golden stripe virus, Peanut clump virus, Potato mop top virus, Rice stripe necrosis virus, Soil-borne wheat mosaic virus, Barley mild mosaic virus, Barley yellow mosaic virus, Oat mosaic virus, Rice necrosis mosaic virus, Wheat spindle streak mosaic virus and Wheat yellow mosaic virus; viruses transmitted via root wound: Tobamovirus genera: Tobacco mosaic virus, Tomato mosaic virus, Cucumber green mottle mosaic tobamovirus, Cucumber fruit mottle mosaic virus, Kyuri green mottle mosaic virus, Odontoglossum ringspot virus, Paprika mild mottle virus, Pepper mild mottle virus, Ribgrass mosaic virus and Tobacco mild green mosaic virus; and
viruses transmitted by unknown rout: Watercress yellow spot virus, Broad been necrotic wilt virus, Peach rosette mosaic virus and Sugarcane chlorotic streak virus.

74. The plant according to claim 71, wherein the at least one segment of the viral genome encodes a putative 54 kDa protein being a fragment of the replication protein of cucumber fruit mottle mosaic virus (CFMMV).

75. The plant according to claim 74, wherein the at least one segment of the viral genome encoding a putative 54 kDa protein has the sequence set forth in SEQ ID NO:1.

76. The plant according to claim 75, wherein the plant is protected from a disease caused by a soil-borne virus of the tobamovirus genus.

77. The plant according to claim 76, wherein the plant is protected from a disease caused by CFMMV.

78. The plant according to claim 77, wherein the plant is of the Cucurbitaceae family.

79. The plant according to claim 78, wherein the plant is a cucumber plant.

80. The plant according to claim 70, wherein the DNA construct comprises nucleic acid sequence encoding an RNA sequence that forms at least one double-stranded RNA molecule, wherein the double stranded RNA molecule mediates cleavage of the at least one segment of the viral genome.

81. The plant according to claim 80, wherein the DNA construct comprises:
a. at least one plant expressible promoter operably linked to;
b. a nucleic acid sequence encoding an RNA sequence that forms at least one double stranded RNA, wherein the double stranded RNA molecule comprises a first nucleotide sequence of at least 20 contiguous nucleotides having at least 90% sequence identity to the sense nucleotide sequence of the target segment of the viral genome and a second nucleotide sequence of at least 20 contiguous nucleotides having at least 90% sequence identity to the complementary sequence of the sense nucleotide sequence of said target segment of said viral genome; and optionally
c. a transcription termination signal.

82. The plant according to claim 81, wherein the first and the second nucleotide sequences are separated by a spacer sequence.

83. The plant according to claim 82, wherein the spacer sequence comprises a sequence of an intron.

84. The plant according to claim 83, wherein the spacer sequence comprises an intron of the castor bean catalase gene, having the sequence set forth in SEQ ID NO:3.

85. The plant according to claim 81, wherein the DNA construct comprises the first and the second nucleotide sequences operably linked to the same promoter.

86. The plant according to claim 80, wherein the plant is resistant to a virus selected from the group consisting of a soil-borne virus and a virus transmitted by a vector affecting the aerial part of the plant.

87. The plant according to claim 86, wherein the soil-borne virus is selected from the group consisting of nematode-transmitted viruses: Nepoviruses: Arabis mosaic virus, Grapevine fanleaf virus, Tomato black ring virus, Raspberry ringspot virus, Tomato ringspot virus, and Tobacco ringspot virus; Tobraviruses: Pea early browning virus, Tobacco rattle virus and Pepper ringspot virus; fungal-transmitted viruses: Cucumber leafspot virus, Cucumber necrosis virus, Melon necrotic spot virus, Red clover necrotic mosaic virus, Squash necrosis virus, Tobacco necrosis satellite virus, Lettuce big-vein virus, Pepper yellow vein virus, Beet necrotic yellow vein virus, Beet soil-borne virus, Oat golden stripe virus, Peanut clump virus, Potato mop top virus, Rice stripe necrosis virus, Soil-borne wheat mosaic virus, Barley mild mosaic virus, Barley yellow mosaic virus, Oat mosaic virus, Rice necrosis mosaic virus, Wheat spindle streak mosaic virus and Wheat yellow mosaic virus; viruses transmitted via root wound: Tobamovirus genera: Tobacco mosaic virus, Tomato mosaic virus, Cucumber green mottle mosaic tobamovirus, Cucumber fruit mottle mosaic virus, Kyuri green mottle mosaic virus, Odontoglossum ringspot virus, Paprika mild mottle virus, Pepper mild mottle virus, Ribgrass mosaic virus and Tobacco mild green mosaic virus; and viruses transmitted by unknown rout: Watercress yellow spot virus, Broad been necrotic wilt virus, Peach rosette mosaic virus and Sugarcane chlorotic streak virus.

88. The plant according to claim 86, wherein the virus transmitted by a vector affecting the aerial parts of the plant is of a family selected from the group consisting of: Caulimoviridae, Geminiviridae, Circoviridae, Reoviridae, Tartitiviridae, Bromoviridae, Comoviridae, Potyviridae, Tombusviridae, Sequiviridae, Clostroviridae and Luteoviridae; Tobamovirus, Tobravirus, Potexvirus, Carlavirus, Allexivirus, Capillovirus, Foveavirus, Trichovirus, Vitivirus, Furovirus, Pecluvirus, Pomovirus, Benyvirus, Hordeivirus, Sobemovirus, Marafivirus, Tymovirus, Idaeovirus, Ourmivirus, Umbravirus.

89. The plant according to claim 80, wherein the at least one segment of the viral genome comprises the 3′ end of Zucchini Yellow Mosaic Virus (ZYMV) genome.

90. The plant according to claim 89, wherein the at least one segment of the viral genome comprises the nucleic acid sequence set forth in SEQ ID NO:2.

91. The plant according to claim 81, wherein the first nucleotide sequence comprises a nucleic acid sequence having at least 90% identity to the nucleotide sequence set forth in SEQ ID NO:2 or fragments thereof.

92. The plant according to claim 91, wherein the second nucleotide sequence comprises a nucleic acid sequence having at least 90% identity to the complement of the nucleotide sequence set forth in SEQ ID NO:2 or fragments thereof.

93. The plant according to claim 92, wherein the plant is resistant to a disease caused by a virus from the Potyviridae family.

94. The plant according to claim 93, wherein the plant is resistant to ZYMV.

95. The plant according to claim 69, wherein the nucleic acid sequence further comprises at least one expression control sequence, selected from the group consisting of a promoter, an enhancer, a transcription factor, a splicing signal, and a termination sequence.

96. The plant according to claim 95, wherein the promoter is a constitutive promoter.

97. The plant according to claim 69, wherein the nucleic acid sequence further comprises a selectable marker.

98. The plant according to claim 97, wherein the selectable marker is selected from a polynucleotide sequence encoding a product conferring antibiotic resistance and a reporter gene encoding a detectable product.

99. The plant according to claim 95, wherein the transcription termination signal is the NOS terminator.

100. A method for producing a plant resistant to a viral disease comprising:
a. providing a transgenic rootstock resistant to the viral disease other than by means of expression of an anti-viral protein;
b. providing a scion susceptible to said viral disease; and
C. grafting the scion onto the rootstock;
thereby obtaining an engrafted plant resistant to the viral disease.

101. The method according to claim 100, wherein the rootstock is transformed with a nucleic acid sequence having at least 90% identity to at least one segment of the viral genome as to produce a transgenic rootstock resistant to the viral disease.

102. The method according to claim 101, wherein the rootstock is transformed with a DNA construct designed for generating siRNAs targeted to the at least one segment of the viral genome.

103. The method according to claim 101, wherein the at least one segment of the viral genome encodes a viral protein or parts thereof, selected from the group consisting of a viral coat protein, a viral replication protein, a viral movement protein or parts thereof.

104. The method according to claim 103, wherein the viral protein is a viral replication protein or part thereof.

105. The method according to claim 104, wherein the transgenic rootstock is resistant to a disease caused by a soil-borne virus, and wherein said plant is protected from a disease caused by a soil-borne virus selected from the group consisting of nematode-transmitted viruses: Nepoviruses: Arabis mosaic virus, Grapevine fanleaf virus, Tomato black ring virus, Raspberry ringspot virus, Tomato ringspot virus, and Tobacco ringspot virus; Tobraviruses: Pea early browning virus, Tobacco rattle virus and Pepper ringspot virus; fungal-transmitted viruses: Cucumber leafspot virus, Cucumber necrosis virus, Melon necrotic spot virus, Red clover necrotic mosaic virus, Squash necrosis virus, Tobacco necrosis satellite virus, Lettuce big-vein virus, Pepper yellow vein virus, Beet necrotic yellow vein virus, Beet soil-borne virus, Oat golden stripe virus, Peanut clump virus, Potato mop top virus, Rice stripe necrosis virus, Soil-borne wheat mosaic virus, Barley mild mosaic virus, Barley yellow mosaic virus, Oat mosaic virus, Rice necrosis mosaic virus, Wheat spindle streak mosaic virus and Wheat yellow mosaic virus; viruses transmitted via root wound: Tobamovirus genera: Tobacco mosaic virus, Tomato mosaic virus, Cucumber green mottle mosaic tobamovirus, Cucumber fruit mottle mosaic virus, Kyuri green mottle mosaic virus, Odontoglossum ringspot virus, Paprika mild mottle virus, Pepper mild mottle virus, Ribgrass mosaic virus and Tobacco mild green mosaic virus; and
viruses transmitted by unknown rout: Watercress yellow spot virus, Broad been necrotic wilt virus, Peach rosette mosaic virus and Sugarcane chlorotic streak virus.

106. The method according to claim 103, wherein the transgenic rootstock comprises a nucleic acid sequence encoding a putative 54 kDa protein being a fragment of the replication protein of cucumber fruit mottle mosaic virus (CFMMV).

107. The method according to claim 106, wherein the transgenic rootstock comprises a nucleic acid sequence encoding a putative 54 kDa protein having the sequence set forth in SEQ ID NO:1.

108. The method according to claim 107, wherein the plant is protected from a disease caused by a soil-borne virus of the tobamovirus genus.

109. The method according to claim 108, wherein the plant is protected from a disease caused by CFMMV.

110. The method according to claim 109, wherein the plant is of the Cucurbitaceae family.

111. The method according to 110, wherein the plant is a cucumber plant.

112. The method according to claim 102, wherein the DNA construct comprises nucleic acid sequence encoding an RNA sequence that forms at least one double-stranded RNA molecule, wherein the double stranded RNA molecule mediates cleavage of the at least one segment of the viral genome.

113. The method according to claim 112, wherein the DNA construct comprises:
a. at least one plant expressible promoter operably linked to;
b. a nucleic acid sequence encoding an RNA sequence that forms at least one double stranded RNA, wherein the double stranded RNA molecule comprises a first nucleotide sequence of at least 20 contiguous nucleotides having at least 90% sequence identity to the sense nucleotide sequence of the target segment of the viral genome and a second nucleotide sequence of at least 20 contiguous nucleotides having at least 90% sequence identity to the complementary sequence of the sense nucleotide sequence of said target segment of said viral genome; and optionally
c. a transcription termination signal.

114. The method according to claim 113, wherein the first and the second nucleotide sequences are separated by a spacer sequence.

115. The method according to claim 114, wherein the spacer sequence comprises a sequence of an intron.

116. The method according to claim 115, wherein the spacer sequence comprises an intron of the castor bean catalase gene, having the sequence set forth in SEQ ID NO:3.

117. The method according to claim 113, wherein the DNA construct comprises the first and the second nucleotide sequences operably linked to the same promoter.

118. The method according to claim 102, wherein the plant is resistant to a virus selected from the group consisting of a soil-borne virus and a virus transmitted by a vector affecting the aerial part of the plant.

119. The method according to claim 118, wherein the soil-borne virus is selected from the group consisting of nematode-transmitted viruses: Nepoviruses: Arabis mosaic virus, Grapevine fanleaf virus, Tomato black ring virus, Raspberry ringspot virus, Tomato ringspot virus, and Tobacco ringspot virus; Tobraviruses: Pea early browning virus, Tobacco rattle virus and Pepper ringspot virus; fungal-transmitted viruses: Cucumber leafspot virus, Cucumber necrosis virus, Melon necrotic spot virus, Red clover necrotic mosaic virus, Squash necrosis virus, Tobacco necrosis satellite virus, Lettuce big-vein virus, Pepper yellow vein virus, Beet necrotic yellow vein virus, Beet soil-borne virus, Oat golden stripe virus, Peanut clump virus, Potato mop top virus, Rice stripe necrosis virus, Soil-borne wheat mosaic virus, Barley mild mosaic virus, Barley yellow mosaic virus, Oat mosaic virus, Rice necrosis mosaic virus, Wheat spindle streak mosaic virus and Wheat yellow mosaic virus; viruses transmitted via root wound: Tobamovirus genera: Tobacco mosaic virus, Tomato mosaic virus, Cucumber green mottle mosaic tobamovirus, Cucumber fruit mottle mosaic virus, Kyuri green mottle mosaic virus, Odontoglossum ringspot virus, Paprika mild mottle virus, Pepper mild mottle virus, Ribgrass mosaic virus and Tobacco mild green mosaic virus; and viruses transmitted by unknown rout: Watercress yellow spot virus, Broad been necrotic wilt virus, Peach rosette mosaic virus and Sugarcane chlorotic streak virus.

120. The method according to claim 118, wherein the virus transmitted by a vector affecting the aerial parts of the plant is of a family selected from the group consisting of: Caulimoviridae, Geminiviridae, Circoviridae, Reoviridae, Tartitiviridae, Bromoviridae, Comoviridae, Potyviridae, Tombusviridae, Sequiviridae, Clostroviridae and Luteoviridae; Tobamovirus, Tobravirus, Potexvirus, Carlavirus, Allexivirus, Capillovirus, Foveavirus, Trichovirus, Vitivirus, Furovirus, Pecluvirus, Pomovirus, Benyvirus, Hordeivirus, Sobemovirus, Marafivirus, Tymovirus, Idaeovirus, Ourmivirus, Umbravirus.

121. The method according to claim 112, wherein the at least one segment of the viral genome comprises the 3′ end of Zucchini Yellow Mosaic Virus (ZYMV) genome.

122. The method according to claim 121, wherein the at least one segment of the viral genome comprises the nucleic acid sequence set forth in SEQ ID NO:2.

123. The method according to claim 113, wherein the first nucleotide sequence comprises a nucleic acid sequence having at least 90% identity to the nucleotide sequence set forth in SEQ ID NO:2 or fragments thereof.

124. The method according to claim 123, wherein the second nucleotide sequence comprises a nucleic acid sequence having at least 90% identity to the complement of the nucleotide sequence set forth in SEQ ID NO:2 or fragments thereof.

125. The method according to claim 124, wherein the plant is resistant to a disease caused by a virus from the Potyviridae family.

126. The method according to claim 125, wherein the plant is resistant to ZYMV.

127. An engrafted plant produced by the method of claim 100.

128. The plant according to claim 127, wherein the plant is resistant to a virus selected from the group consisting of a soil-borne virus and a virus transmitted by a vector affecting the aerial part of the plant.

129. The plant according to claim 128, wherein the plant is resistant to a soil-borne virus selected from the group consisting of nematode-transmitted viruses:
Nepoviruses: Arabis mosaic virus, Grapevine fanleaf virus, Tomato black ring virus, Raspberry ringspot virus, Tomato ringspot virus, and Tobacco ringspot virus;
Tobraviruses: Pea early browning virus, Tobacco rattle virus and Pepper ringspot virus;
fungal-transmitted viruses: Cucumber leafspot virus, Cucumber necrosis virus, Melon necrotic spot virus, Red clover necrotic mosaic virus, Squash necrosis virus, Tobacco necrosis satellite virus, Lettuce big-vein virus, Pepper yellow vein virus, Beet necrotic yellow vein virus, Beet soil-borne virus, Oat golden stripe virus, Peanut clump virus, Potato mop top virus, Rice stripe necrosis virus, Soil-borne wheat mosaic virus, Barley mild mosaic virus, Barley yellow mosaic virus, Oat mosaic virus, Rice necrosis mosaic virus, Wheat spindle streak mosaic virus and Wheat yellow mosaic virus; viruses transmitted via root wound: Tobamovirus genera: Tobacco mosaic virus, Tomato mosaic virus, Cucumber green mottle mosaic tobamovirus, Cucumber fruit mottle mosaic virus, Kyuri green mottle mosaic virus, Odontoglossum ringspot virus, Paprika mild mottle virus, Pepper mild mottle virus, Ribgrass mosaic virus and Tobacco mild green mosaic virus; and viruses transmitted by unknown rout: Watercress yellow spot virus, Broad been necrotic wilt virus, Peach rosette mosaic virus and Sugarcane chlorotic streak virus.

130. The plant according to claim 128, wherein the plant is resistant to a virus transmitted by a vector affecting the aerial parts of the plant of a family selected from the group consisting of: Caulimoviridae, Geminiviridae, Circoviridae, Reoviridae, Tartitiviridae, Bromoviridae, Comoviridae, Potyviridae, Tombusviridae, Sequiviridae, Clostroviridae and Luteoviridae; Tobamovirus, Tobravirus, Potexvirus, Carlavirus, Allexivirus, Capillovirus, Foveavirus, Trichovirus, Vitivirus, Furovirus, Pecluvirus, Pomovirus, Benyvirus, Hordeivirus, Sobemovirus, Marafivirus, Tymovirus, Idaeovirus, Ourmivirus, Umbravirus.
